(19) United States
(12) Patent Application Publication
(10) Pub. No.: US 2020/0188431 A1
(43) Pub. Date: Jun. 18, 2020

(54) GLUTAMINE DEHYDROGENASE INHIBITORS FOR USE IN MUSCLE REGENERATION

(71) Applicants: VIB VZW, Gent (BE); KATHOLIEKE UNIVERSITEIT LEUVEN, K.U.LEUVEN R&D, Leuven (BE)

(72) Inventors: Massimiliano Mazzone, Kessel-Lo (BE); Min Shang, Heverlee (BE)

(21) Appl. No.: 16/620,082

(22) PCT Filed: Jun. 8, 2018
(86) PCT No.: PCT/EP2018/065125

§ 371 Date: Dec. 6, 2019

(30) Foreign Application Priority Data
Jun. 9, 2017 (EP) 17175210.8
Feb. 12, 2018 (EP) 18156309.9

(51) Int. Cl. A61K 35/15; A61K 45/06; C12N 15/113; A61P 21/00

(52) U.S. Cl. CPC A61K 35/15; A61P 21/00; C12N 15/1137; A61K 45/06

(57) ABSTRACT

The present invention relates to the field of muscle pathologies, more particularly to the field of diseases where skeletal muscle wasting occurs. The invention provides the use of inhibitors of glutamine dehydrogenase for the regeneration of skeletal muscle.

### FIELD OF THE INVENTION

The present invention relates to the field of muscle pathologies, more particularly to the field of diseases where skeletal muscle wasting occurs. The invention provides the use of inhibitors of glutamine dehydrogenase for the regeneration of skeletal muscle.

### BACKGROUND OF THE INVENTION

Skeletal muscles are composed of bundles of highly oriented and dense muscle fibers, each a multinucleoated cell derived from myoblasts. The muscle fibers in native skeletal muscle are closely packed together in an extracellular three-dimensional matrix to form an organized tissue with high cell density and cellular orientation to generate longitudinal contraction. After muscle injuries, myofibers become necrotic and are removed by macrophages. A specialized myoblast sub-population called satellite cells (SCs) scattered below the basal lamina of myofibers are capable of regeneration. The incidence of satellite cells in skeletal muscle is very low (1%-5%) and depends on age and muscle fiber composition. These cells remain in a quiescent and undifferentiated state and can enter the mitotic cycle in response to specific local factors. This induces proliferation and fusion of myoblasts to form multinucleated and elongated myotubes, which self-assemble to form a more organized structure, namely muscle fiber. Besides satellite cells migrate and proliferate in the injured area and can form a connective tissue network (muscle fibrosis). This process is called “scar tissue formation” and leads to loss of functionality. Several diseases often result in significant loss of skeletal muscle tissue. Examples include skeletal myopathies such as muscular dystrophy and spinal muscular atrophy. In addition traumatic injury, aggressive tumor ablation and prolonged denervation lead to skeletal muscle loss. Until now, few alternatives exist to provide functional and aesthetic restoration of lost muscle tissues aside from transfer of muscle from local or distant sites. However, the tissue engineering of skeletal muscle tissue still remains a challenge. Exploring cost-effective interventions that are able to maintain muscle mass, muscle strength, and physical performance during muscle damage and loss is an important public health challenge (Argiles et al. 2016, J Am Med Dir Assoc 17:789-796), particularly taking into consideration that: (i) diabetes, hypercholesterolemia, obesity are recurrent conditions in our society that are often associated with cardiovascular problems (Grover et al. 2015, Lancet Diabetes Endocrinol 3:114-122); (ii) age-related sarcopenia is even more relevant in today's world given the increased life expectancy and the progressively aging population (Argiles et al. 2016, J Am Med Dir Assoc 17:789-796). Research aiming to reduce morbidity and to increase the life quality of this large fraction of world population needs to be boosted. Surgical interventions and stent design, together with protocols aiming to revascularize the necrotic muscle are the most advanced strategies in case of ischemia (Liebert et al. 2017, Sci Rep 7:42386). In the aging field, therapeutic innovations are hardly seen. Among all the feasible approaches, insulin-like growth factor-1 (IGF-1) has emerged as a growth factor with a remarkably wide range of actions and a tremendous potential to attenuate the atrophy and fragility associated with muscle aging and diseases (Vinciguerra et al. 2010, Adv Exp Med Biol 694:211-233). Although it is now accepted that SC depletion in aged mice worsens muscle regeneration without affecting sarcopenia (Fry et al. 2015, Nat Med 21:76-80), the inverse scenario that a gain-of-function approach for SC activity prevents muscle loss/sarcopenia in absence of injury is very intriguing. So far, only one report has shown that SC transplantation counteracts age-related impairment in muscle mass, function and strength (Hall et al. 2010, Sci Transl Med 2:57ra83). In addition, a number of other extracellular factors are known to be involved in the muscle regeneration that is triggered in response to muscle damage. Some of them, such as fibroblast growth factors (FGFs), hepatocyte growth factor (HGF), transforming growth factor (TGF)-like molecules, leukemia inhibitor factor (LIF) or platelet-derived growth factors (PDGFs), are involved in the activation of cell proliferation that operates before muscle differentiation. In addition, factors such as IGFs, neuregulins (NRGs), sonic hedgehog (Shh) or Wnt promote muscle differentiation. However up till now none of these molecules have proven to be useful for muscle regeneration therapy and it is clear that a more detailed understanding of the signalling pathways triggered by these factors is needed.

Muscle regeneration is the process by which damaged skeletal, smooth or cardiac muscle undergoes biological repair and formation of new muscle in response to death (necrosis) of muscle cells. The success of the regenerative process depends upon the extent of the initial damage and many intrinsic and environmental factors. Key cellular events required for regeneration include inflammation, revascularisation and innervation, in addition to myogenesis where new muscle is formed. In mammals, new muscle formation is generally excellent for skeletal muscle but poor for cardiac muscle; however a greater capacity for regeneration of cardiac muscle is seen in fish and some anurans. Muscle regeneration recapitulates many aspects of embryonic myogenesis and is an important homeostatic process of the adult skeletal muscle, which, after development, retains the capacity to regenerate in response to appropriate stimuli, activating the muscle compartment of stem cells, namely, satellite cells, as well as other precursor cells. Moreover, significant evidence suggests that while stem cells represent an important determinant for tissue regeneration, a “qualified” environment is necessary to guarantee and achieve functional results. Skeletal muscle injury and regeneration are closely associated with macrophages, which have a pro-inflammatory M1-like phenotype or anti-inflammatory M2-like phenotype. Metabolically, M1 macrophages are glycolytic, while M2 macrophages are oxidative. However, it remains unknown if a change in macrophage metabolism effects a specific macrophages phenotype and how this affects skeletal muscle regeneration.

### SUMMARY OF THE INVENTION

In one aspect, the invention relates to an inhibitor of GLUD1 for use in treating muscle wasting, muscle wasting disease, muscle atrophy, muscle injury, or muscle insult in a subject. The muscle injury or insult may for instance be of ischemic or traumatic origin.

In another aspect, the invention relates to an inhibitor of GLUD1 for use in preventing, inhibiting, ameliorating or halting muscle wasting, muscle wasting disease or muscle atrophy in an elderly subject, in an immobile subject, or in a subject at risk of developing muscle wasting, muscle wasting disease, or muscle atrophy.

In a further aspect, the invention relates to an inhibitor of GLUD1 for use in activating muscle satellite cells in an elderly subject, in an immobile subject, or in a subject at risk of developing muscle wasting, muscle wasting disease, or muscle atrophy.

In any of the above, the muscle wasting, muscle wasting disease, or muscle atrophy may be sarcopenia. The muscle wasting, muscle wasting disease, or muscle atrophy may alternatively be associated with any of cachexia, cancer, AIDS, coeliac disease, chronic obstructive pulmonary disease (COPD), multiple sclerosis (MS), arthrititis, rheumatoid arthritis (RA), congestive heart failure, tuberculosis (TBC), familial amyloid polyneuropathy, mercury poisoning (acrodynia), Crohn's disease, untreated/severe type 1 diabetes mellitus, anorexia nervosa, hormonal deficiency, frailty syndrome, spinal muscle atrophy, stroke, steroid therapy, poliomyelitis, spinal cord injury, hypercatabolic disease, or myotonia congenital.

In any of the above, the inhibitor of GLUD1 may be a small compound inhibiting GLUD1, a nucleic acid based inhibitor of GLUD1, a biopharmaceutical compound inhibiting GLUD1, a GLUD1 knock-out macrophage, or a macrophage conditionally expressing a GLUD1 inhibitor, wherein said macrophage (in practice more than one or a population of macrophages) may be transferred to the subject. In particular, the nucleic acid based inhibitor may be a GLUD1-selective nucleic acid based inhibitor selected from a gapmer, a shRNA, a siRNA, an artificial microRNA, a dsRNA, an anti-sense oligomer, a ribozyme, a morpholino, a locked nucleic acid, a peptide nucleic acid, a Zinc-finger nuclease, a TALEN, a CRISPR-Cas, a CRISPR-C2c2, or a meganuclease. More in particular, the nucleic acid based inhibitor of GLUD 1 may be an RNA based inhibitor of GLUD1. Alternatively, the inhibitor of GLUD1 may be a small compound inhibiting GLUD1 and may be selected from the group consisting of R162, purpurin, aurintricarboxylic acid, hexachlorophene, GW5074, bithionol, CK2 inhibitor, BSB, leoidin, erythrosin B, metergoline, diethylstilbestrol, calmidazolium, BH3I-2, suloctidil, ethaverine hydrochloride, epigallocatechin 3,5,-digallate, epigallocatechin 3-monogallate, epicatechin 3-monogallate, epigallocatechin, epicatechin, gallic acid, dimethyl-α-ketoglutarate, hymecromone methyl ether, strophanthidin, allopurinol, 5,7-dihydroxy-methylcoumarin, a compound according to any of Formulas I-VI as described hereinafter, and GLUD1-inhibiting analogues of any of the foregoing compounds; or selected from the group consisting of a prodrug, co-crystal, polymorph and salt of any of the foregoing compounds or analogues.

The invention further relates to pharmaceutical compositions comprising a GLUD1 inhibitor for any of the above uses. Such pharmaceutical composition may further comprise an excipient.

The invention also provides an isolated GLUD1 knock-out macrophage, or an isolated macrophage conditionally expressing a GLUD1 inhibitor for use as a medicament (such as for administering to a subject, e.g. cell transfer or adoptive cell transfer); as well as pharmaceutical compositions comprising an isolated GLUD1 knock-out macrophage or an isolated macrophage conditionally expressing a GLUD1 inhibitor. Such pharmaceutical composition may further comprise an excipient.

### DESCRIPTION TO THE FIGURES

FIG. 1. GLUD1 deficiency in macrophages reduces skeletal muscle damage.

a-c: Quantification (a-b) and representative images (c) of necrotic and regenerating tissue on hematoxylin and eosin (H&E)-stained sections from tibialis anterior (TA) muscles 6 days after cardiotoxin (CTX) injury. The lower right corner, confined by the dotted line, and the black arrowhead show necrotic fibers; the upper left corner, confined by the dotted line, displays an area of regenerating fibers. Data show representative values for 3 independent experiments (10 samples per condition per experiment).

d: Voluntary wheel running test before (from day −2 to day 0) and after cardiotoxin (CTX) injury (from day 1 to day 10). CTX was administered at day 0 immediately after recording the basal running distance. A dotted line highlights the moment of CTX injection in both right and left crural muscles (in two different points i.e., in the TA and in the gastrocnemius muscles). The analysis was performed on 5 CTRL vs 5 Glud1^{ΔMo} mice. e-g: Quantification (e-f) and representative images (g) of necrotic and regenerating tissue on H&E stained sections from crural muscles 14 days after femoral artery ligation. Data show representative values for 3 independent experiments (number of samples included in the analysis: CTRL=9; Glud1^{ΔMo}=6).

h-k: Quantification and representative images of TUNEL⁺ cells (h-i) and DHE⁺ area (j-k) in TA muscle 6 days after CTX injury. Data show representative values for 3 independent experiments. Number of samples included in the analysis: CTRL, n=7; Glud1^{ΔMo}, n=8 (h-i); CTRL, n=4; Glud1^{ΔMo}, n=4 (j-k).

l-o: Quantification and representative images of TUNEL⁺ cells (l-m) and DHE⁺ area (o-p) in crural muscles 14 days after ligation. Data show representative values for 3 independent experiments (4 samples per condition per experiment).

p-r: Quantification (p-q) and representative images (r) of necrotic and regenerating tissue on H&E stained sections from TA muscles 6 days after CTX injury following inducible macrophage-specific Glud1 deletion (Csf1r; Glud1^{L/L}); tamoxifen-injected littermates were used as controls (Csf1r; Glud1^{WT/WT}). 6 samples per condition per experiment.

*P<0.05; **P<0.005; ***P<0,0005; ****P<0,00005. Scale bars: 10 μm (i); 20 μm (k, m, o); 50 μm (c, g, r). Graphs show mean±standard error of the mean (SEM).

FIG. 2. GLUD1 deficiency in macrophages induces muscle regeneration by activating SC.

a: Quantification of necrotic area on H&E stained sections from TA muscles 1 day and 6 days after CTX injury. Data show representative values from at least 2 independent experiments (number of samples included in the analysis: PBS, n=4; Day1, n=6; Day6, n=10).

b: Quantification of necrotic area on H&E stained sections from crural muscles 1 day, 3 days and 14 days after ligation. Data show representative values from at least 2 independent experiments. Number of samples included in the analysis: Baseline, n=4; Day1, n=5; Day3, n=8; Day14, n=10.

c-d: Quantification of F4/80⁺ area (c) in TA muscle 1 day and 6 days after CTX injury. Representative images of F4/80⁺ area (d) in TA muscle 6 days after CTX injury. Data show representative values from at least 2 independent experiments (number of samples included in the analysis: PBS, n=4; Day1, n=6; Day6, n=10).

e-f: Quantification of F4/80⁺ area (e) in crural muscles 1 day, 3 days and 14 days after ligation. Representative images of F4/80⁺ area (f) in crural muscles 14 days after ligation. Data show representative values from at least 2 independent experiments (number of samples included in the analysis: baseline, n=4; Day1, n=5; Day3, n=8; Day14, n=10).

g: Quantification of crystal-violet-stained WT or GLUD1 KO bone-marrow derived macrophages (BMDMs), migrating towards a gradient of serum (20% FBS). Data show representative values from 3 independent experiments (the graph shows values of 3 biological repetitions per condition).

h: Laser Doppler analysis 1, 3, 6, 9 and 13 days post-ligation. The analysis was performed on 5 CTRL vs 5 Glud1^{ΔMo} mice.

i: Quantification of vessel numbers in crural muscles 14 days after ligation. Data show representative values for 2 independent experiments (5 samples per condition per experiment).

j-k: Quantification of proliferating muscle precursors (j) in TA muscle 1 day and 6 days after CTX injury. Representative images of proliferating muscle precursors (k) in TA muscle 1 day after CTX injury. White arrows indicate PHH3⁺ proliferating cells, adjacent to the laminin⁺ basal lamina.

l-m: Quantification of proliferating muscle precursors (l) in crural muscles 1 day, 3 days and 14 days after ligation. Representative images of proliferating muscle precursors (m) in crural muscles 3 days after ligation.

n-o: Western blot for PAX 7 in TA muscle lysates from CTRL or Glud1^{ΔMo} mice 1 day after CTX injury. Vinculin was used as loading control. Numbers represent densitometric fold change towards CTRL. Data show representative values for 3 independent experiments.

p-q: Western blot for PAX 7 in crural muscle lysates from CTRL or Glud1^{ΔMo} mice 3 days after ligation. Vinculin was used as loading control. Numbers represent densitometric fold change towards CTRL. Data show representative values for 3 independent experiments.

r-s: Quantification (r) and representative images (s) of single fibers and analysis of total nuclei associated to freshly isolated single fibers from CTRL or Glud^{ΔMo} mice 1 day after CTX injury. The analysis was performed on 5 CTRL vs 5 Glud1^{ΔMo} mice.

*P<0.05; **P<0.005; ***P<0,0005; ns, not significant (P>0.05). Scale bars: 20 μm (d, f, s); 50 μm (k, m). Graphs show mean±standard error of the mean (SEM).

In bar graphs (a, b, c, e, g, i, j, l, n, p, r), results with controls (CTRL) or wild-type (WT) are compared under a certain condition (indicated on the x-axis) to results with Glud1ΔMo or GLUD1 KO, respectively. In these graphs, and per test condition, the left bars refer to controls or wild-type whereas the right bars refer to Glud1ΔMo or GLUD1 KO.

FIG. 3. GLUD1 deficiency in macrophages promotes glutamine production.

a: Glutamine oxidation in BMDMs cultured in glutamine-enriched and glutamine-reduced media.

b: Quantification (by GC-MS) of intracellular 2-oxoglutarate content from BMDMs cultured in glutamine-enriched and glutamine-reduced media.

c-d: Quantification (by GC-MS) of intracellular (c) and extracellular (d) glutamine content from BMDMs cultured in glutamine-enriched and glutamine-reduced media.

e: [U-¹⁴C]-glutamine uptake in BMDMs cultured in glutamine-enriched and glutamine-reduced media.

f: Evaluation of the [U-¹³C]-glutamine-derived carbon incorporation levels into glutamate from BMDMs.

g: Evaluation of glutamine syntethase (GS) activity by analysing the percentage of the ¹⁵NH4⁺-derived ammonia incorporation levels into glutamine from BMDMs.

h-i: WB analysis (h) and quantification of GS (i) protein levels in BMDMs cultured in glutamine-enriched media and glutamine-reduced media. Vinculin was used as loading control. Numbers represent densitometric fold change towards CTRL.

j-k: WB analysis (j) and quantification of GLUD1 (k) protein levels in BMDMs cultured in glutamine-enriched and glutamine-reduced media. Vinculin was used as loading control. Numbers represent densitometric fold change towards CTRL.

l: Evaluation of the [U-¹³C]-glucose-derived carbon incorporation levels into 2-oxoglutarate from BMDMs cultured in glutamine-enriched and glutamine-reduced media.

m-n: Evaluation of the [U-¹³C]-glucose-derived (m) and [¹⁵N,¹³C4]-aspartate-derived (n) incorporation levels into glutamate from BMDMs.

o: Quantification (by GC-MS) of extracellular glutamine content from BMDMs under glutamine deprivation treated with and without MSO.

All experiments, data show representative values for 3 independent experiments (per condition per experiment, n>=3). *P<0.05; **P<0.005; ns, not significant (P>0.05). Graphs show mean±standard error of the mean (SEM).

In bar graphs (a, b, c, d, e, g, h, j, l, o), results with wild-type (WT) are compared under a certain condition (indicated on the x-axis) to results with GLUD1 KO. In these graphs, and per test condition, the left bars refer to wild-type whereas the right bars refer to GLUD1 KO.

FIG. 4. Glutamine release by GLUD1-deficient macrophages promotes SC activation and muscle regeneration.

a-b: Quantification (a) and representative images (b) of myotube formation in C2C12 cells co-cultured with BMDMs. Average myotube area was measured in presence of glutamine-enriched and glutamine-reduced media. The graph shows values of 3 biological repetitions per condition.

c-d: Quantifications (c) and representative images (d) of myotube formation in C2C12 cells cultured with BMDM-conditioned media (CM). The graph shows values of 3 biological repetitions per condition.

e: [U-¹⁴C]-glutamine uptake in SLC1A5-deficient C2C12 cells obtained by co-expressing the Cas9 along with a gRNA targeting the Slc1a5 locus. Wild type cells (Ctrl) and a non-targeting scrambled (Scrl) gRNA were used as negative controls. C2C12 cells treated with SLC1A5 inhibitor gamma-L-Glutamyl-p-Nitroanilide (GPNA) was used as a positive control. The graph shows values of 3 biological repetitions per condition.

f-g: Quantification (f) and representative images (g) of myotube formation in SLC1A5-deficient C2C12 cells co-cultured with BMDMs under glutamine deprivation. Scramble (Scrl) C2C12 cells were used as a control. The graph shows values of 3 biological repetitions per condition.

h: Glutamine to glutamate ratio in the interstitial fluid of TA muscle 1 day after CTX injury. Data show representative values for 2 independent experiments (number of samples included in the analysis: PBS, n=5; CTX, n=9).

i: Glutamine to glutamate ratio in the interstitial fluid of crural muscles 3 days after ligation. Data show representative values for 2 independent experiments (number of samples included in the analysis: baseline, n=9; ligated, n=12).

j: Quantification (by GC-MS) of extracellular glutamate content from WT or GLUD1 KO BMDMs in glutamine-enriched and glutamine-reduced media. Data show representative values for 3 independent experiments (3 biological repetitions are included in the analysis).

k: Glutamate to leucine ratio in the interstitial fluid of TA muscles 1 day after CTX injury.

l: Quantifications of necrotic area on H&E stained sections from TA muscles 6 days after CTX injury. Number of samples included in the analysis: Glud1^{ΔMo} GS^{WT}, n=8; Glud^{ΔMo} GS^{WT}, n=6; Glud1^{WT} GS^{ΔMo}, n=3; Glud1^{ΔMo} GS^{ΔMo}, n=8.

m: Quantification of proliferating muscle precursors on sections of TA muscles 1 day after CTX injury, stained for PHH3 and laminin. CTRL and Glud1^{ΔM0} mice were treated 3 times per day with the SLC1A5 inhibitor GPNA, or vehicle as control. Number of samples included in the analysis: CTRL with vehicle, n=5; Glud1^{ΔMo} with vehicle, n=5; CTRL with GPNA, n=8; Glud1^{ΔMo} with GPNA, n=8.

*P<0.05; **P<0.005; ***P<0,0005; ns, not significant (P>0.05). Scale bars: 50 μm (b, d, g). Graphs show mean±standard error of the mean (SEM).

In bar graphs (a, c, f), results with C2C12 only, C2C12+WT and C2C12+GLUD1 KO are compared under a certain condition (indicated on the x-axis). In these graphs, and per test condition, the left bars refer to C2C12 only, the middle bars refer to C2C12+WT, and the right bars refer to C2C12+GLUD1 KO.

In bar graphs (h, l, j, k, m), results with controls (CTRL) or wild-type (WT) are compared under a certain condition (indicated on the x-axis) to results with Glud1ΔMo or GLUD1 KO, respectively. In these graphs, and per test condition, the left bars refer to controls or wild-type whereas the right bars refer to Glud1ΔMo or GLUD1 KO.

FIG. 5. Pharmacologic blockade of GLUD1 promotes muscle regeneration.

a-c: Quantification of regenerating fibers (a) and intact differentiated fibers (b) on H&E-stained sections (c) from TA muscles 6 days after CTX injury. Wild type mice were bi-daily treated with R162, or vehicle as control.

d-e: Quantification (d) and representative images (e) of F4/80⁺ area in TA muscles, 6 days after CTX injury. Wild type mice were bi-daily treated with R162, or vehicle as control.

f-g: Quantification (f) and representative images (g) of proliferating muscle precursors on sections of TA muscles 1 day after CTX injury, stained for PHH3 and laminin. Wild type mice were bi-daily treated with R162, or vehicle as control.

h-j: Quantification of the necrotic area (h) and intact differentiated fibers (i) on H&E-stained sections (j) from crural muscles 14 days after ligation. Wild type mice were bi-daily treated with R162, or vehicle as control.

k-l: Quantification (k) and representative images (l) of F4/80⁺ area in crural muscles, 14 days after ligation. Wild type mice were bi-daily treated with R162, or vehicle as control.

All experiments, the analysis was performed on 5 mice per condition. *P<0.05; **P<0.005. Scale bars: 50 μm (c, e, g, j, l). Graphs show mean±standard error of the mean (SEM).

In bar graphs (a, b, d, f, h, l, k), results with Vehicle or Baseline are compared under a certain condition (indicated on the x-axis) to results with R162 or Ligated, respectively. In these graphs, and per test condition, the left bars refer to Vehicle or Baseline whereas the right bars refer to R162 or Ligated.

FIG. 6. GLUD1 deficiency in macrophages ameliorates age-related muscle wasting.

a-c: Grip strength test (a), rotarod test (b) and voluntary wheel running activity (c) in aged mice (16-18 months). The analysis was performed on 5 CTRL vs 5 Glud1^{ΔMo} mice.

d-e: Quantification of necrotic (d) crural muscles and representative images (e) on H&E stained sections in aged mice. The analysis was performed on 6 CTRL vs 6 Glud1^{ΔMo} mice.

f-g: Masson's trichrome staining of crural muscle sections proves the absence of collagen deposition in young mice but its appearance (dark blue fibers and connective tissue) in aged mice though to a much less extent in Glud1^{ΔMo} mice (f). Morphometric quantification of blue area over total muscle area in Masson's trichrome stained crural muscle sections from CTRL and Glud1^{ΔMo} aged mice (g). Number of samples included in the analysis: young mice, CTRL=6, Glud1^{ΔMo}=6; aged mice, CTRL=5; Glud1^{ΔMo}=8.

h: Quantification of F4/80+ area in crural muscles of aged mice. Number of samples included in the analysis: CTRL=8; Glud1^{ΔMo}=8.

i-j: Quantification (i) and representative images (j) of single fibers and analysis of total nuclei associated to freshly isolated fibers in aged mice. Number of samples included in the analysis: CTRL=8; Glud1^{ΔMo}=7.

k-l: Quantifications (k) and representative images (l) of Pax7⁺ SC (adjacent to the laminin+basal lamina) in TA muscles of aged mice. White arrows indicate Pax7⁺ cells. Number of samples included in the analysis: CTRL=5; Glud1^{ΔMo}=6.

m: Muscle index in aged mice obtained by dividing the weight of the gastrocnemius muscle for the body weight. Number of samples included in the analysis: young mice, CTRL=8, Glud1^{ΔMo}=8; aged mice, CTRL=6; Glud1^{ΔMo}=9.

n: Scheme illustrating the role of Glud1 in macrophages during muscle regeneration. Muscle tissue represents one of the major sources of glutamine. During acute muscle damage, ischemia or aging, glutamine can result limiting, especially due to the fact that infiltrating macrophages by means of GLUD1 upregulation can utilize glutamine and enter in competition with activated satellite cells (SC), the latter highly benefiting from glutamine uptake for their functions. Because of this competition, glutamine availability for SC is restrained which impairs muscle regeneration. By pharmacologic or genetic targeting of GLUD1 in macrophages, accumulation of glutamate promotes the induction of glutamine synthetase. Instead of being used by macrophages, excessive glutamine is secreted into the extracellular milieu which favours SC proliferation. The increased pool of SC will then enhance muscle regeneration and prevent muscle wasting.

*P<0.05. Scale bars: 20 μm (e, j), 50 μm (f, l). Graphs show mean±standard error of the mean (SEM).

In bar graphs (m), results with controls (CTRL) are compared under a certain condition (indicated on the x-axis) to results with Glud1ΔMo. In these graphs, and per test condition, the left bars refer to controls whereas the right bars refer to Glud1ΔMo.

FIG. 7. GLUD1-inhibitors

a: purpurin (1,2,4-trihydroxyanthraquinone)

b: R162, a purpurin analog (2-allyl-1-hydroxy-9,10-anthraquinone, 2-allyl-1-hydroxyanthracene-9,10-dione), an analog of purpurin

FIG. 8. GLUD1 expression in macrophages of wild-type and Glud1ΔMo mice.

(a) Representative image of Western blot for the Glud1 in BMDMs isolated from wild-type (CTRL) and Glud1ΔMo (GLUD1 KO) mice. Representative of 3 independent Western blots.

(b) qRT-PCR for the Glud1 in F4/80⁺ macrophages, freshly sorted after 5-day in vivo treatment with tamoxifen (n=3). Number of mice included in the analysis: wild-type (CTRL), 4; Glud1ΔMo (L/L), 4.

**P<0.005. Graphs show mean±standard error of the mean (SEM).

FIG. 9. Expression of phenotypic markers in wild-type and Glud1ΔMo macrophages.

(a-d) qRT-PCR for the Cxcl9 (a), TNFa (b), Arg1 (c) and IL10 (d) in BMDMs isolated from wild-type (CTRL) and Glud1ΔMo (GLUD1 KO) mice. The graph shows values of three biological repetitions per condition.

(e-h) FACS analysis of different M1 (MHC II high, CD80) or M2 (MHC II low, CD206) polarization markers in CD45+CD11b+F4/80⁺ macrophages isolated from TA muscle lysate of wild-type (CTRL) and Glud1ΔMo (GLUD1 KO) mice 1 day after CTX injury. Number of mice included in the analysis: CTRL, 5; Glud1ΔMo, 5.

ns, not significant (P>0.05). Graphs show mean±standard error of the mean (SEM).

In bar graphs (e, f, g, h), results with controls (CTRL) are compared under a certain condition (indicated on the x-axis) to results with Glud1ΔMo. In these graphs, and per test condition, the left bars refer to controls whereas the right bars refer to Glud1ΔMo.

FIG. 10. Energetic markers in wild-type and Glud1ΔMo macrophages.

(a-b) LC-MS measurement of total cellular energy charge ([ATP]+1/2[ADP]/[ATP]+[ADP]+[AMP]) (b) and ATP content (a) in BMDMs. The graph shows values of three biological repetitions per condition.

(c) Oxygen consumption rate (OCR) in BMDMs. The graph shows values of three biological repetitions per condition.

not significant (P>0.05). Graphs show mean±standard error of the mean (SEM).

In bar graphs (a, b, c), results with wild-type (WT) are compared under a certain condition (indicated on the x-axis) to results with Glud1ΔMo (GLUD1 KO), respectively. In these graphs, and per test condition, the left bars refer to wild-type whereas the right bars refer to Glud1ΔMo.

FIG. 11. Slc1a5 expression in SLC1A5-deficient C2C12 cells, and interstitial muscle fluid glutamate in wild-type and Glud1ΔMo mice

(a) qRT-PCR for the Slc1a5 in SLC1A5-deficient C2C12 cells obtained by co-expressing the Cas9 along with a gRNA targeting the Slc1a5 locus. The graph shows values of three biological repetitions per condition.

(b) Extracellular abundance of glutamate as determined in the interstitial fluid from muscles of control (CTRL) mice or Glud1ΔMo mice.

***P<0,0005. Graphs show mean±standard error of the mean (SEM).

In bar graphs (b), results with controls (CTRL) are compared under a certain condition (indicated on the x-axis) to results with Glud1ΔMo. In these graphs, and per test condition, the left bars refer to controls whereas the right bars refer to Glud1ΔMo.

### DETAILED DESCRIPTION OF THE INVENTION

In work leading to the present invention the knockout of glutamine dehydrogenase (GLUD1) in macrophages was investigated and it was reasoned that this would prevent glutamine anaplerosis and thus utilization of this amino acid by macrophages. It was observed that the extracellular milieu was enriched with glutamine in view of a strong induction of glutamine synthetase (GS). Surprisingly it was shown that the release of glutamine by GLUD1-deficient macrophages strongly favors satellite cell (SC) activation, proliferation and differentiation towards muscle cells. It was further demonstrated that pharmacological inhibitors of GLUD1 not only prevented ischemic necrosis but also activated satellite cells in support of muscle regeneration. Therefore, macrophage-specific deletion of GLUD1 or pharmacologic inhibition of GLUD1 promotes myogenic differentiation, prevents muscle wasting and improves functional recovery upon acute or chronic damages such as toxin-induced myolysis, ischemia or aging.

The role of macrophages during muscle regeneration has been previously elucidated (Kharraz et al. 2013, Mediators Inflamm 2013:491497; Saclier et al. 2013, Stem Cells 31:384-396; Tidball 2017, Nat Rev immunol 17:165-178; Zhang et al. 2013, J Biol Chem 288:1489-1499; Zhang et al. 2009, Am J Pathol 175:2518-2527; Arnold et al. 2007, J Exp Med 204:1057-1069). It is well known in the art that, following acute injury, macrophages strongly contribute to the repair of the damaged skeletal muscle (Kharraz et al. 2013, Mediators Inflamm 2013:491497; Saclier et al. 2013, Stem Cells 31:384-396; Tidball 2017, Nat Rev immunol 17:165-178; Zhang et al. 2009, Am J Pathol 175:2518-2527; Arnold et al. 2007, J Exp Med 204:1057-1069). Macrophages clear tissue debris and release cytokines as well as growth factors that stimulate myoblast proliferation (Kharraz et al. 2013, Mediators Inflamm 2013:491497; Zhang et al. 2013, J Biol Chem 288:1489-1499; Zhang et al. 2009, Am J Pathol 175:2518-2527). At a later stage, macrophages promote myoblast differentiation and fusion (Arnold et al. 2007, J Exp Med 204:1057-1069), but also contribute to tissue revascularization (Latroche et al. 2017, Stem Cell Rep 9:2018-2033). If muscle degeneration persists, such as during sarcopenia, macrophage infiltration perpetuates and leads to progressive loss of muscle mass and function (Wang et al. 2015, Aging Cell 14:678-688). The positive involvement of inflammatory cells in the acute phase of muscle healing is further supported by the evidence that macrophage depletion impairs muscle regenerative capacity (Bentzinger et al. 2013, EMBO Rep 14:1062-1072; Lemos et al. 2015, Nat Med 21:786-794; Munoz-Canoves & Serrano 2015, Trends Endocrin Met 26:449-450; Summan et al. 2006, Am J Physiol Regul Integr Comp Physiol 290:R1488-1495). In the present invention, it was shown via the inhibition of the metabolic enzyme GLUD1, in both acute and chronic settings, that macrophages (the most represented leukocyte population, infiltrating the damaged muscle) enter in competition with satellite cells (SC cells) for glutamine, which becomes a limiting substrate for satellite activation under muscle degeneration (see FIG. 6n). Muscle tissue is a major site for glutamine synthesis in the human body. In most physiologic conditions, myocytes will release glutamine that goes in circulation but also fills the parenchymal interstitium (Biolo et al. 1995, Am J Physiol 268:E75-84; Nurjhan et al. 1995, J Clin Invest 95:272-277). However, in some pathologic conditions muscle tissue may reduce glutamine output (Newsholme & Parry-Billings 1990, J Parenter Enteral Nutr 14:63S-67S). For example, it was shown that muscle regeneration upon cardiotoxin-induced myolysis, femoral artery occlusion, and during aging, is retrained by limited glutamine availability, probably emphasized not only by the lack of glutamine output due to the damaged muscle itself but, in some cases, also by a poor blood supply, given the fact that glutamine is the most concentrated amino acid in the blood (Stumvoll et al. 1999, Kidney Int 55:778-792). In these conditions, the deleterious competition between macrophages and SC is broken upon GLUD1 deletion in macrophages. Interestingly, glutamine uptake in absence of GLUD1 remains the same. However, the impossibility to direct glutamate to the TCA cycle in absence of GLUD1 boosts the induction of glutamine synthetase which permits the cells to channel excessive, cytotoxic glutamate towards glutamine production and excretion. The contribution of glutamine as energy and nitrogen source is well-compensated in macrophages. It was indeed proven that transaminase activity is higher in GLUD1-knockout macrophages suggesting that the usage of nitrogen can be anyhow sustained in case of reduced glutaminolysis, while the amount of glutamine-derived 2-oxoglutarate is countered by the increased use of alternative carbon sources e.g., glucose or fatty acids. On the other hand, glutamine is known to play an important role in muscle precursors (Girven et al. 2016, J Cell Physiol 231:2720-2732; Smith et al. 1984, J Cell Physiol 120:197-203). It constitutes the basis for the production of glutathione which is relevant to protect proliferating cells by oxidative stress (Kozakowska et al. 2015, J Muscle Res Cell Motil 36:377-393). It also constitutes an important brick to build muscle fibers (Rennie et al. 1989, Metabolism 38:47-51). Finally, glutamine activates mTOR (Jewell et al. 2015, Science 347:194-198) which unleashes SC proliferation and growth (Rodgers et al. 2014, Nature 510:393-396; Zhang et al. 2015, Biochem Biophys Res Commun 463:102-108).

Biologically, the findings described in this invention are intriguing because they emphasize the relevance of a close cross-talk between inflammatory cells and SC. In this regard, local availability of glutamine is more important for SC than systemic glutamine concentrations. This highlights the metabolic role of a muscle stem cell niche as it has been shown in other physiological and pathological contexts such as in the brain (Quaegebeur et al. 2016, Cell Metab 23:280-291) or in cancer (Liao et al. 2017, Mol Cancer 16:51). Based on the data presented herein, increased glutamine production by GLUD1-deficient macrophages suggests that glutamine synthesis is not only reinforced when the end-product (i.e., glutamine) is poorly present in the microenvironment (Palmieri et al. 2017, Cell Rep 20:1654-1666) but also when the substrate for this reaction (i.e., glutamate) is in excess, as in the absence of GLUD1.

The experiments described herein pave the way towards a novel possibility to treat skeletal muscle ischemia and other types of muscle degenerations, as well as muscle loss in aging patients. By using GLUD1 inhibitors (exemplified herein by R162 previously shown to be safely used in mouse models of cancer therapy; Palmieri et al. 2017, Cell Rep 20:1654-1666), it was shown that GLUD1 inhibitors can be used in the treatment of muscle wasting disorders. Besides the use of small molecules that inhibit GLUD1, adoptive transfer of GLUD1-knockout macrophages can also be considered in case of muscle wasting. The latter strategy would avoid to block GLUD1 systemically and particularly in the SC compartment, although from our data and others (Ryall et al. 2015, Cell Stem Cell 16:171-183), it emerges that SC can compensate for the absence of GLUD1. In addition, the data presented herein suggest that chronic use of GLUD1 blockers in elderly people may prevent muscle loss, reduce risk of injuries and improve their quality of life.

In conclusion, it was shown that glutamine restriction in case of acute and chronic muscle damage, can invalidate the positive role of macrophages during tissue repair. By a metabolic reprogramming of macrophages, the competition between SC and infiltrating macrophages for glutamine is broken, and macrophages are enforced to supply glutamine and nurse SC, thus installing a positive metabolic cross-talk between these two compartments.

The wording ‘diseased skeletal muscle cells’ refers to skeletal muscle cells that have been exposed for example to an ischemic insult, or for example skeletal muscle cells that possess a reduced glycolytic rate, or for example skeletal muscle cells that have been exposed to serum deprivation. ‘Degeneration’ is herein equivalent to the terms necrotic skeletal muscle cell death, apoptotic muscle skeletal cell death, muscle skeletal cell atrophy, skeletal fiber injury and skeletal muscle wasting. The term ‘skeletal muscle degenerative diseases’ or ‘skeletal muscle wasting diseases’ comprises any of a group of diseases where degeneration (atrophy) occurs of skeletal muscle cells or diseases where structural changes or functional impairment occur in skeletal muscle. A serious indication where skeletal muscle degeneration takes place is due to ischemic insults or traumatic insults/injuries. For example it has become increasingly recognized that skeletal muscle atrophy is common in patients with chronic pulmonary disease (COPD). Another example where skeletal muscle atrophy occurs is critical limb ischemia (CLI) which is a disease manifested by sharply diminished blood flow to the legs. Up to 10 million people in the US alone suffer from severe leg pain (claudication) and non-healing-ulcers (peripheral vascular disease), both of which can ultimately lead to CLI. The most common causes that can lead to CLI are atherosclerosis and embolization (e.g. a clot that has been ejected from a failing heart, or from an aneurysm in the aorta, into the leg). Yet another class of skeletal muscle degenerative diseases are muscle pathologies associated with a reduced glycolytic rate such as McArdle's disease and phosphofructokinase disease (PFKD). Yet another class comprises muscle atrophy which occurs due to muscle denervation. In such denervation atrophy, there occurs a lack of tonic stimuli and muscle cells become atrophic. Causes of denervation atrophy include localized loss of nerve function (neuritis) or generalized loss of the entire motor unit. After denervation, muscles become rapidly atrophic and 50% of muscle mass could be lost in just a few weeks. Examples are peripheral motor neuropathies and motoneuron disorders such as amyotrophic lateral sclerosis, Guillain-Barré syndrome and diabetic neuropathy. Another class of such diseases comprises muscle degeneration which occurs due to immobilization. ‘Immobilization’ means here that the skeletal muscle system is unloaded because of for example prolonged space flight (an astronauts disease), during conservative treatment after sports injuries, in bedridden or otherwise (chronically) immobile subjects, or by a plaster cast after orthopedic surgery. This immobilization causes a serious atrophy of muscle mass leading to a decrease in physical performance and high power output capacity. Yet another class of such diseases where muscle degeneration takes place comprises muscular dystrophies. These disorders include a progressive wasting of skeletal muscle. The most common examples are Duchenne and Becker muscular dystrophy. Yet another class of conditions were muscle degeneration takes place comprises critical illness. Critical illness (e.g. burns, sepsis) is associated with a serious muscle wasting and muscle weakness. In addition, muscle wasting also occurs in cachexia (wasting syndrome, e.g. which frequently occurs in cancer patients), in aging related sarcopenia, in cachexia-related or cachexia-associated sarcopenia, in spinal muscle atrophy, in arthritis, in stroke, in steroid therapy, in poliomyelitis, in spinal cord injury, in hypercatabolic disease, and in myotonia congenita. Sarcopenia in general is a degenerative skeletal muscle mass and strength loss or muscle atrophy often accompanied by a reduction of muscle tissue quality (replacement of muscle mass by fat, increased fibrosis, degeneration of neuromuscular junction, changes in muscle metabolism, increased oxidative stress), and often a component of/associated with the frailty syndrome.

Cachexia is frequently occurring in subjects suffering from cancer, AIDS, coeliac disease, chronic obstructive pulmonary disease (COPD), multiple sclerosis (MS), rheumatoid arthritis (RA), congestive heart failure, tuberculosis (TBC), familial amyloid polyneuropathy, mercury poisoning (acrodynia), Crohn's disease, untreated/severe type 1 diabetes mellitus, anorexia nervosa and hormonal deficiency. Muscle wasting, muscle wasting disease, muscle atrophy or sarcopenia thus can be associated with cachexia or any such diseases such as cancer, AIDS, coeliac disease, chronic obstructive pulmonary disease (COPD), multiple sclerosis (MS), rheumatoid arthritis (RA), congestive heart failure, tuberculosis (TBC), familial amyloid polyneuropathy, mercury poisoning (acrodynia), Crohn's disease, untreated/severe type 1 diabetes mellitus, anorexia nervosa and hormonal deficiency.

Satellite cells, or muscle satellite cells, are mononuclear cells that are normally activated upon injury or exercise. Activation of satellite cells may be followed by their proliferation and/or differentiation and fusion into the muscle fiber, therewith supporting or maintaining muscle function. Age-related muscle wasting, muscle atrophy or sarcopenia is reviewed by e.g. Ryall et al. 2008 (Biogerontology 9:213-228).

In view of all of the above, the invention therefore relates, in a first aspect, to a GLUD1 inhibitor for use in treating or (in a method of) treatment of muscle atrophy, muscle injury or insult, muscle wasting, muscle wasting disease, myopathy, or muscle wasting or muscle atrophy associated with a disease (which may be other than a muscle or muscular disease) in a subject. The muscle injury or insult may for instance be of ischemic or traumatic origin. The muscle wasting, muscle wasting disease, or muscle atrophy may be sarcopenia. Alternatively, the muscle wasting, muscle wasting disease, or muscle atrophy may be associated with any of cachexia, cancer, AIDS, coeliac disease, chronic obstructive pulmonary disease (COPD), multiple sclerosis (MS), arthrititis, rheumatoid arthritis (RA), congestive heart failure, tuberculosis (TBC), familial amyloid polyneuropathy, mercury poisoning (acrodynia), Crohn's disease, untreated/severe type 1 diabetes mellitus, anorexia nervosa, hormonal deficiency, frailty syndrome, spinal muscle atrophy, stroke, steroid therapy, poliomyelitis, spinal cord injury, hypercatabolic disease, or myotonia congenital.

The invention in another aspect relates to a GLUD1 inhibitor for use in (a method of) preventing, inhibiting, ameliorating or halting muscle wasting, muscle wasting disease, muscle atrophy, or myopathy in an elderly subject, in an immobile subject; or in a subject at risk of developing muscle wasting, muscle wasting disease, muscle atrophy, or myopathy.

In a further aspect, the invention also relates to a GLUD1 inhibitor for use in (a method of) activating muscle satellite cells (SCs) in an elderly subject, in an immobile subject, or in a subject at risk of developing muscle wasting, muscle wasting disease, muscle atrophy, or myopathy. The effect of the GLUD1 inhibitor on SC cells may in particular be an activating effect, an effect on proliferation (stimulating proliferation), an effect on differentiation (stimulating differentiation, in particular differentiation to muscle cells or myocytes), or any combination of any of these effects; activation of SCs for instance may result in their proliferation and/or differentiation. In particular, the effect of the GLUD1 inhibitor on activation and proliferation and/or differentiation of SCs is underlying and common to the therapeutic and prophylactic uses of the GLUD1 inhibitor as contemplated herein.

Furthermore, the methods referred to hereinabove may comprise the step of administering to a subject a therapeutically or prophylactically effective amount of GLUD1 inhibitor or of a pharmaceutical composition comprising a GLUD1 inhibitor. Therewith the therapeutic or prophylactic effect as contemplated herein is obtained.

In one embodiment the invention provides an inhibitor of functional expression of GLUD1 for use in the above-described aspects. In another particular embodiment a small compound inhibiting the function of GLUD1 is provided for use in the above-described aspects is provided. In a more particular embodiment, a nucleotide based inhibitor of GLUD1 is provided. In an even more particular embodiment, an RNA based inhibitor of GLUD1 is provided. In particular embodiments, said inhibitor or nucleotide based inhibitor or RNA based inhibitor is a GLUD1-selective inhibitor selected from a gapmer, a shRNA, a siRNA, an artificial microRNA, a dsRNA, an anti-sense oligomer, a ribozyme, a morpholino, a locked nucleic acid, a peptide nucleic acid, a Zinc-finger nuclease, a TALEN, a CRISPR-Cas, a CRISPR-C2c2 or a meganuclease.

In a further embodiment the invention provides a biopharmaceutical inhibitor of GLUD1 for use in the above-described aspects. Such biopharmaceutical agents for inhibiting GLUD1 activity include (monoclonal) antibodies or antigen-binding fragments thereof, alpha-bodies, nanobodies, intrabodies (antibodies binding and/or acting to intracellular target; this typically requires the expression of the antibody within the target cell, which can be accomplished by gene therapy), aptamers, DARPins, affibodies, affitins, anticalins, and monobodies (see further).

In yet a further embodiment the invention provides macrophages wherein the expression of the GLUD1 gene is knocked-out or otherwise downregulated (see further) as inhibitor of GLUD1 for use in the above-described aspects. In particular, such macrophages are for use in a treatment (therapeutic or prophylactic) comprising transfer or adoptive transfer of the macrophages to a subject.

With “GLUD1-selective inhibitor” is meant an inhibitor that is either fully selective to GLUD1 and is not inhibiting targets other than GLUD1. GLUD1-selective inhibitors further include those inhibitors which are highly selective to GLUD1 compared to other targets. For example, any candidate GLUD1-inhibitor which is inhibiting GLUD1 activity e.g. at least 5-fold, at least 5- to 10-fold, at least 10-fold, or at least 10-fold or higher, more strongly than another target is considered to be a GLUD1-selective inhibitor. The comparison can be one e.g. comparing IC50-values (concentration of compound required to inhibit 50% of target activity). Methods for determining (residual) activity of GLUD are known in the art, such as e.g. described in Jin et al. 2015 (Cancer Cell 27:157-270).

GLUD1 (HGNC: 4335, Entrez Gene: 2746, Ensembl: ENSG00000148672, OMIM: 138130 and UniProtKB: P00367), also known as glutamate dehydrogenase 1, but herein further designated as glutamine dehydrogenase 1 (EC 1.4.1.3.). The enzyme is a mitochondrial matrix enzyme that catalyzes the oxidative deamination of glutamate to alpha-ketoglutarate and ammonia. This enzyme has an important role in regulating amino acid-induced insulin secretion. It is allosterically activated by ADP and inhibited by GTP and ATP. The human nucleic acid sequence which encodes the GLUD1 protein is depicted in SEQ ID NO:1, in particular this is the mRNA sequence encoding human GLUD1 (GenBank accession number M20867.1). Other reference sequence mRNAs/transcript variants include those provided in Genbank accession numbers NM_001318900.1, NM_001318901.1, NM_001318902.1, NM_001318904.1, NM_001318905.1, NM_001318906.1, NM_005271.4. The GLUD1 protein amino acid sequence corresponding to SEQ ID NO:1 is depicted in SEQ ID NO:2. These sequences provide a basis for searching for related sequences such as (splicing) variants or gene sequences (e.g. using the Basic Local Alignment Search Tool or BLAST).

Glutaminolysis is a mitochondrial pathway that involves the initial deamination of glutamine by glutaminase (GLS), yielding glutamate and ammonia. Glutamate is then converted to alpha-ketoglutarate (alpha-KG), a TCA cycle intermediate, to produce both ATP and anabolic carbons for the synthesis of amino acids, nucleotides, and lipids. The conversion of glutamate to alpha-KG is catalyzed by either glutamine dehydrogenase 1 (GDH1, also known as GLUD1, GLUD, GDH or glutamate dehydrogenase). In addition, other transaminases, including glutamate pyruvate transaminase 2 (GPT2, also known as alanine aminotransferase), and glutamate oxaloacetate transaminase 2 (GOT2, also known as aspartate aminotransferase), can convert alpha-keto acids into their corresponding amino acids in mitochondria. The flux of GLUD1 is commonly elevated in human cancers. Alpha-KG, a product of GDH1 and a key intermediate in glutamine metabolism, is known to stabilize redox homeostasis in cells. Although elevated glutaminolysis and altered redox status in cancer cells has been theoretically justified, the mechanism by which alpha-KG regulates redox and whether this regulation is crucial for tumorigenesis and tumor growth remain elusive.

In humans, GLUD1 is located on Chromosome 10: start: 87,050,202 bp from pter End: 87,095,019 bp from pter, Orientation: minus strand. In particular embodiments, GLUD1 is defined by the nucleic acid sequence that shows at least 90%, at least 91%, at least 92%, at least 93%, at least 94%, at least 95%, at least 96%, at least 97%, at least 98%, at least 99% identity to SEQ ID NO:1, to a splicing variant of SEQ ID NO:1, or to a GLUD1 transcript variant (such as listed above). In a particular embodiment, an inhibitor of functional expression or a nucleotide based inhibitor or an RNA based inhibitor of a nucleic acid sequence is provided, wherein said nucleic acid sequence encodes SEQ ID NO:1. In another particular embodiment, an inhibitor of functional expression or a nucleotide based inhibitor or an RNA based inhibitor of a nucleic acid sequence that shows at least 90%, at least 91%, at least 92%, at least 93%, at least 94%, at least 95%, at least 96%, at least 97%, at least 98%, at least 99% identity to SEQ ID NO:, to a splicing variant of SEQ ID NO:1, or to a GLUD1 transcript variant (such as listed above) is provided. In another particular embodiment, an inhibitor of functional expression or a nucleotide based inhibitor or an RNA based inhibitor of SEQ ID NO:1, of a splicing variant of SEQ ID NO:1, or of a GLUD1 transcript variant (such as listed above) is provided.

The “sequence identity” of two related nucleotide or amino acid sequences, expressed as a percentage, refers to the number of positions in the two optimally aligned sequences which have identical residues (×100) divided by the number of positions compared. A gap, i.e., a position in an alignment where a residue is present in one sequence but not in the other is regarded as a position with non-identical residues. The alignment of the two sequences is performed by the Needleman and Wunsch algorithm (Needleman and Wunsch (1970) J Mol Biol. 48: 443-453). The computer-assisted sequence alignment above, can be conveniently performed using standard software program such as GAP which is part of the Wisconsin Package Version 10.1 (Genetics Computer Group, Madison, Wis., USA) using the default scoring matrix with a gap creation penalty of 50 and a gap extension penalty of 3. Sequences are indicated as “essentially similar” when such sequences have a sequence identity of at least about 75%, particularly at least about 80%, more particularly at least about 85%, quite particularly about 90%, especially about 95%, more especially about 100%, quite especially are identical.

With “functional expression”, in the present application it is meant the transcription and/or translation of functional gene product. For the GLUD1 protein coding genes “functional expression” can be deregulated on at least two levels. First, at the DNA level, e.g. by absence or disruption of the gene, or lack of transcription taking place (in both instances preventing synthesis of the relevant gene product). The lack of transcription can e.g. be caused by epigenetic changes (e.g. DNA methylation) or by loss-of-function mutations. A “loss-of-function” or “LOF” mutation as used herein is a mutation that prevents, reduces or abolishes the function of a gene product as opposed to a gain-of-function mutation that confers enhanced or new activity on a protein. LOF can be caused by a wide range of mutation types, including, but not limited to, a deletion of the entire gene or part of the gene, splice site mutations, frame-shift mutations caused by small insertions and deletions, nonsense mutations, missense mutations replacing an essential amino acid and mutations preventing correct cellular localization of the product. Also included within this definition are mutations in promoters or regulatory regions of the GLUD1 gene if these interfere with gene function. A null mutation is an LOF mutation that completely abolishes the function of the gene product. A null mutation in one allele will typically reduce expression levels by about 50%, but may have severe effects on the function of the gene product. Note that functional expression can also be deregulated because of a gain-of-function mutation: by conferring a new activity on the protein, the normal function of the protein is deregulated, and less functionally active protein is expressed. Vice versa, functional expression can be increased e.g. through gene duplication or by lack of DNA methylation.

Second, at the RNA level, e.g. by lack of efficient translation taking place—e.g. because of destabilization of the mRNA (e.g. by UTR variants) so that it is degraded before translation occurs from the transcript. Or by lack of efficient transcription, e.g. because a mutation introduces a new splicing variant.

The above may collectively be referred to as to knocking out or knock out of GLUD1 gene expression or GLUD1 expression.

On yet another level the function activity (or the enzymatic activity) of the GLUD1 protein can be inhibited. Several small molecule inhibitors are described of GLUD1. For example US20160228466 (Emory University) discloses specific GLUD1 small molecule inhibitors in FIG. 6A. One particular compound which has been used in the instant invention is R162 (analog-3 disclosed in US20160228466). The same purpurin analogues are disclosed by Jin et al. 2015 (Cancer Cell 27:257-270); US20160228466 discloses GLUD1 inhibitors in claims 2 to 6. Jin et al. 2015 (Cancer Cell 27:257-270) also identified other candidate GLUD1 inhibitors from a library of 2000 FDA-approved small molecule compounds i.e. hymecromone methyl ether (7-methoxy-4-methylcoumarin; 7-methoxy-4-methyl-2H-chromen-2-one; 2H-1-benzopyran-2-one, 7-methoxy-4-methyl-; 4-methylherniarin), strophanthidin (convallatoxigenin; strophanthidine; corchsularin; corchorgenin; corchoside A aglycon; 3 beta,5,14-Trihydroxy-19-oxo-5 beta-card-20(22)-enolide), allopurinol (1,5-Dihydro-4H-pyrazolo(3,4-d)pyrimidine-4-one), and 5,7-dihydroxy-methylcoumarin. Close analogues of these compounds with GLUD1-inhibitory activity, e.g. purpurin, are also included for the purpose of the current invention. Any of the above compounds, as well as their close analogues thereof, in the form of a prodrug, co-crystal, polymorph or salt are also included for the purpose of the current invention.

In particular, a pharmacological GLUD1 inhibitor has the structure of Formula I, or is a prodrug, co-crystal, polymoroph, derivative, or salt thereof:

[symbol]

wherein,

X is hydroxyl, optionally substituted with R¹⁰;

R¹ is C₁₋₆ alkenyl, wherein R¹ is optionally substituted with one or more, the same or different, R¹⁰;

R², R³, R⁴, R⁵, R⁶, and R⁷ are each individually and independently hydrogen, alkyl, halogen, nitro, cyano, hydroxy, amino, mercapto, formyl, carboxy, carbamoyl, alkoxy, alkylthio, alkylamino, (alkyl)₂amino, alkanoyl, alkylsulfinyl, alkyl sulfonyl, aryl sulfonyl, benzyl, benzoyl, carbocyclyl, aryl, aryloxy, or heterocyclyl, wherein R², R³, R⁴, R⁵, R⁶, and R⁷ are optionally substituted with one or more, the same or different, R¹⁰;

R¹⁰ is alkyl, halogen, nitro, cyano, hydroxy, amino, mercapto, formyl, carboxy, carbamoyl, alkoxy, alkylthio, alkylamino, (alkyl)₂amino, alkanoyl, alkylsulfinyl, alkylsulfonyl, arylsulfonyl, benzyl, benzoyl, carbocyclyl, aryl, aryloxy, or heterocyclyl, wherein R¹⁰ is optionally substituted with one or more, the same or different, R¹¹;

and

R¹¹ is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethyl sulfonyl, methoxycarbonyl, ethoxycarbonyl, N-methylsulfamoyl, N-ethylsulfamoyl, N,N-dimethyl sulfamoyl, N,N-diethyl sulfamoyl, N-methyl-N-ethylsulfamoyl, benzyl, benzoyl, carbocyclyl, aryl, or heterocyclyl.

In particular, X may be hydroxyl and R¹ may be an allyl.

Yet other small molecule inhibitors are described in WO2017084598 (Hangzhou Gamma Biotech Co. Ltd), the latter application discloses selenium-containing inhibitors binding to an allosteric site of GLUD1. Further in particular, a pharmacological GLUD1 inhibitor has the structure of any one of the Formulas II-V, or is a prodrug, co-crystal, polymoroph, derivative, or salt thereof:

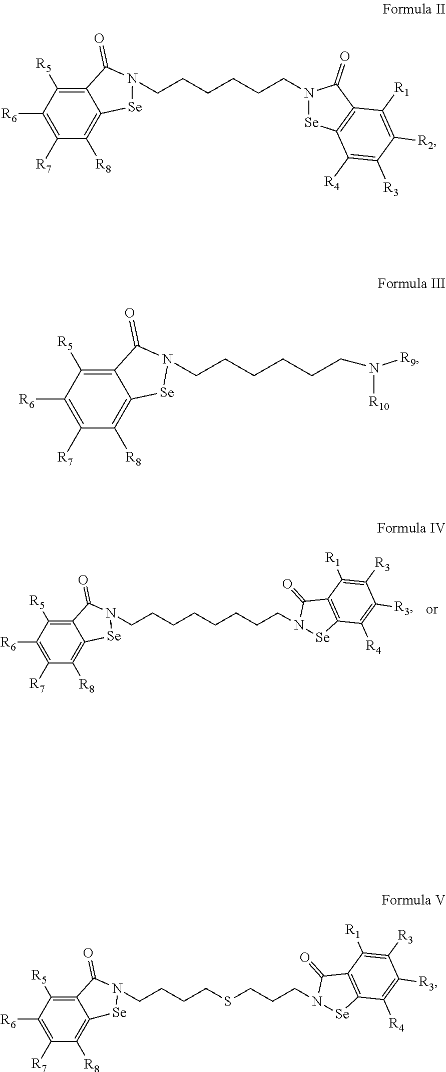

wherein:

R1, R2, R3, R4, R5, R6, R7, R8, R9 and R10 are independently selected substituents comprising 1-20 atoms selected independently from the group of C, H, N, O, S, P, Si, and halogen. In one embodiment, at least one of R1, R2, R3, and R4 are not hydrogen substituents, and/or at least one of R5, R6, R7, and R8 are not hydrogen substituents. In a further embodiment, R1, R2, R3, R4, R5, R6, R7, R8 are H. In yet a further embodiment, R1, R2, R3, R4, R5, R6, R7, R8 are substituents selected independently from an aromatic heterocycle, a heterocycle, a substituted alkyl, an amide, an ether, a halogen, a silane, a thioether, an amine, a phosphate, a sulfoxide, a sulfonyl. In a further embodiment, R9 is unsubstituted and R10 is a substituent comprising 1-20 atoms selected independently from the group of C, H, N, O, S, P, Si, and halogen. In a further embodiment, R9 is unsubstituted, and R10 is a substituent selected from the group of an aromatic heterocycle, a heterocycle, a substituted alkyl, an amide, an ether, a halogen, a silane, a thioether, an amine, a phosphate group, a sulfoxide, a sulfonyl and related derivatives thereof. Further in particular, a pharmacological GLUD1 inhibitor has the structure of any one of the Formula VI, or is a prodrug, co-crystal, polymoroph, derivative, or salt thereof:

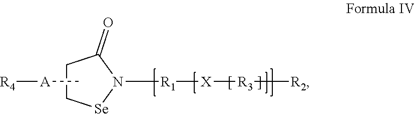

wherein:

R1 and R3 are substituents independently selected from the group comprising straight-chain alkyl, branched alkyl, C3-C6 cycloalkyl, aromatic, and 3-5 membered heterocyclic substituents; A, which contains one or more independently selected R4 groups which locate in any substitution positions from A, is selected from the group comprising mono- or polycyclic heteroaryl fused with a selenazole or cyclo-alkyl;

X is selected from C, N, S, Se or O;

R2 is unsubstituted, or is substituted by substituent W1;

R4 is selected from H and substituent W2;

wherein substituent W1 and substituent W2 are chemically stable groups or substituents;

wherein the heterocyclic substituent is a heterocyclic functional group or substituent comprising one or more heteroatoms independently selected from N, S, O, Se; and

wherein the polycyclic heteroaryl is a functional group or substituent fused by heterocyclic substituent and/or monocyclic aryl.

In one embodiment, W1 and W2 may be independently selected substituents comprising 1-10 atoms independently selected from the group of C, H, N, O, S, P, halogen; for example independently selected from straight-chain alkyl, branched alkyl, cycloalkyl, heterocyclic substituent, polycyclic heteroaryl, amide, ether, lipid, halogen, silane, thioether, amine, phosphate group, sulfoxide, sulfonyl and related derivatives thereof. In a further embodiment, R1 and R3 are independently selected from functional groups or substituents comprising 4 or more C. In a further embodiment, R2 is a C1-C8 substituent selected from the group of straight-chain alkyl, branched alkyl, cycloalkyl, aryl, heterocyclic substituent, and polycyclic heteroaryl. In one particular embodiment, R2 is comprising benzoisoselenazol-3-ketone. Yet further pharmacological or small molecule inhibitors of GLUD1 include those disclosed by Li et al. 2007 (Biochemistry 46:15089-15102): aurintricarboxylic acid, hexachlorophene, GW5074, bithionol, CK2 inhibitor, BSB, leoidin, erythrosin B, metergoline, diethylstilbestrol, calmidazolium, BH3I-2, suloctidil, ethaverine hydrochloride, epigallocatechin 3,5,-digallate, epigallocatechin 3-monogallate, epicatechin 3-monogallate, epigallocatechin, epicatechin and gallic acid. GLUD1 inhibitors used by Polletta et al. 2015 (Autophagy 11:253-270) include hexachlorophene and dimethyl-α-ketoglutarate.

It is envisaged that these small molecule inhibitors can be used in handling muscle wasting disorders as described in the aspects of the invention. In particular, such small molecule inhibitor of GLUD1 is selected from the group consisting of R162, purpurin, aurintricarboxylic acid, hexachlorophene, GW5074, bithionol, CK2 inhibitor, BSB, leoidin, erythrosin B, metergoline, diethylstilbestrol, calmidazolium, BH3I-2, suloctidil, ethaverine hydrochloride, epigallocatechin 3,5,-digallate, epigallocatechin 3-monogallate, epicatechin 3-monogallate, epigallocatechin, epicatechin, gallic acid, dimethyl-α-ketoglutarate, hymecromone methyl ether, strophanthidin, allopurinol, 5,7-dihydroxy-methylcoumarin, a compound according to any of Formulas I-VI, and GLUD1-inhibiting analogues of any of the foregoing compounds; or selected from the group consisting of a prodrug, co-crystal, polymorph and salt of any of the foregoing compounds or analogues.

In the present application it is essential that the functional expression of GLUD1 or the functional activity of GLUD1 is inhibited in order to have a positive/healing effect on muscle wasting disorders. The inhibition of the functional expression of GLUD1 or the inhibition of the functional activity of GLUD1 is preferably at least 30%, at least 40%, at least 50%, at least 60%, at least 70%, at least 80%, at least 90%, at least 95% or even 100% compared to a control situation wherein the functional expression of GLUD1 is not inhibited. 100% means that no detectable functional expression or no detectable functional activity of GLUD1. The nature, route of administration, and dosing or dosing regimen of the GLUD1-inhibiting compound is not vital/essential to the invention as long as the GLUD1 activity is sufficiently inhibited such as to arrive at the envisaged therapeutic or prophylactic effect.

Gene inactivation, i.e. inhibition of functional expression of the target gene, can be achieved through transfection of cell in in vitro conditions, administration of a RNA- or DNA-based inhibitor to cells, the creation of transgenic organisms expressing antisense RNA, or by administering antisense RNA to the subject. An antisense construct targeting GLUD1 can be delivered, for example, as an expression plasmid, which, when transcribed in the cell, produces RNA that is complementary to at least a unique portion of the cellular GLUD1 RNA.

An inhibitor of functional expression of the GLUD1 genes can also be an antisense molecule or anti-gene agent that comprises an oligomer of at least about 10 nucleotides in length for which no transcription is needed in the treated subject. In embodiments such an inhibitor comprises at least 15, 18 20, 25, 30, 35, 40, or 50 nucleotides. Antisense approaches involve the design of oligonucleotides (either DNA or RNA, or derivatives thereof) that are complementary to an RNA encoded by polynucleotide sequences of the GLUD1 gene. Antisense RNA may be introduced into a cell to inhibit translation of a complementary mRNA by base pairing to it and physically obstructing the translation machinery. This effect is therefore stoichiometric. Absolute complementarity, although preferred, is not required. A sequence “complementary” to a portion of an RNA, as referred to herein, means a sequence having sufficient complementarity to be able to hybridize with the RNA, forming a stable duplex; in the case of double stranded antisense polynucleotide sequences, a single strand of the duplex DNA may thus be tested, or triplex formation may be assayed. The ability to hybridize will depend on both the degree of complementarity and the length of the antisense polynucleotide sequence. Generally, the longer the hybridizing polynucleotide sequence, the more base mismatches with an RNA it may contain and still form a stable duplex (or triplex, as the case may be). One skilled in the art can ascertain a tolerable degree of mismatch by use of standard procedures to determine the melting point of the hybridized complex. Antisense oligomers should be at least 10 nucleotides in length, and are preferably oligomers ranging from 15 to about 50 nucleotides in length. In certain embodiments, the oligomer is at least 15 nucleotides, at least 18 nucleotides, at least 20 nucleotides, at least 25 nucleotides, at least 30 nucleotides, at least 35 nucleotides, at least 40 nucleotides, or at least 50 nucleotides in length. A related method uses ribozymes instead of antisense RNA. Ribozymes are catalytic RNA molecules with enzyme-like cleavage properties that can be designed to target specific RNA sequences. Successful target gene inactivation, including temporally and tissue-specific gene inactivation, using ribozymes has been reported in mouse, zebrafish and fruitflies.

RNA interference (RNAi) is a form of post-transcriptional gene silencing and used in this application as one of the many methods to inhibit or reduce the functional expression of GLUD1. The phenomenon of RNA interference was first observed and described in Caenorhabditis elegans where exogenous double-stranded RNA (dsRNA) was shown to specifically and potently disrupt the activity of genes containing homologous sequences through a mechanism that induces rapid degradation of the target RNA. Numerous reports have describe the same catalytic phenomenon in other organisms, including experiments demonstrating spatial and/or temporal control of gene inactivation, including plants, protozoa, invertebrates, vertebrates and mammals. RNAi mediated degradation of the target mRNA can be detected by measuring levels of the target mRNA or protein in the cells of a subject, using standard techniques for isolating and quantifying mRNA or protein as described in this application. The mediators of sequence-specific messenger RNA degradation are small interfering RNAs (siRNAs) generated by ribonuclease III cleavage from longer dsRNAs. Generally, the length of siRNAs is between 20-25 nucleotides (Elbashir et al. 2001 Nature 411, 494 498). The siRNA typically comprise a sense RNA strand and a complementary antisense RNA strand annealed together by standard Watson Crick base pairing interactions (hereinafter “base paired”). The sense strand comprises a nucleic acid sequence that is identical to a target sequence (i.e. of the GLUD1 sequence in this application) contained within the target mRNA. The sense and antisense strands of the present siRNA can comprise two complementary, single stranded RNA molecules or can comprise a single molecule in which two complementary portions are base paired and are covalently linked by a single stranded “hairpin” area (often referred to as shRNA). The siRNAs that can be used to inhibit or reduce the functional expression of lipin can comprise partially purified RNA, substantially pure RNA, synthetic RNA, or recombinantly produced RNA, as well as altered RNA that differs from naturally occurring RNA by the addition, deletion, substitution and/or alteration of one or more nucleotides. Such alterations can include addition of non-nucleotide material, such as to the end(s) of the siRNA or to one or more internal nucleotides of the siRNA, including modifications that make the siRNA resistant to nuclease digestion. The siRNAs can be targeted to any stretch of approximately 19 to 25 contiguous nucleotides in the GLUD1 sequence (the “target sequence”). Techniques for selecting target sequences for siRNA are well known in the art. Thus, the sense strand of the present siRNA comprises a nucleotide sequence identical to any contiguous stretch of about 19 to about 25 nucleotides in the target mRNA. siRNAs can be obtained using a number of techniques known to those of skill in the art. For example, the siRNAs can be chemically synthesized or recombinantly produced using methods known in the art. Preferably, the siRNA of the invention are chemically synthesized using appropriately protected ribonucleoside phosphoramidites and a conventional DNA/RNA synthesizer. The siRNA can be synthesized as two separate, complementary RNA molecules, or as a single RNA molecule with two complementary regions. Commercial suppliers of synthetic RNA molecules or synthesis reagents include Proligo (Hamburg, Germany), Dharmacon Research (Lafayette, Colo., USA), Pierce Chemical (part of Perbio Science, Rockford, Ill., USA), Glen Research (Sterling, Va., USA), ChemGenes (Ashland, Mass., USA) and Cruachem (Glasgow, UK). Alternatively, siRNA can also be expressed from recombinant circular or linear DNA plasmids using any suitable promoter. Suitable promoters for expressing siRNA targeted against GLUD1 expression from a plasmid include, for example, the U6 or H1 RNA pol III promoter sequences and the cytomegalovirus promoter. Selection of other suitable promoters is within the skill in the art. The recombinant plasmids of the invention can also comprise inducible or regulatable promoters for expression of the siRNA in a particular tissue or in a particular intracellular environment. The siRNA expressed from recombinant plasmids can either be isolated from cultured cell expression systems by standard techniques, or can be expressed intracellularly, e.g. in tumor tissue, cells with the eye or in tissue that are prone to inflammation. siRNAs can also be expressed intracellularly from recombinant viral vectors. The recombinant viral vectors comprise sequences encoding the siRNAs of the invention and any suitable promoter for expressing the siRNA sequences. The siRNA will be administered in an “effective amount” which is an amount sufficient to cause RNAi mediated degradation of the target mRNA. One skilled in the art can readily determine an effective amount of the siRNA of the invention to be administered to a given subject, by taking into account factors such as involuntary muscle contraction; the extent of the disease penetration; the age, health and sex of the subject; the route of administration; and whether the administration is regional or systemic. Generally, an effective amount of siRNAs targeting GLUD1 gene expression comprises an intracellular concentration of from about 1 nanomolar (nM) to about 100 nM, preferably from about 2 nM to about 50 nM, more preferably from about 2.5 nM to about 10 nM. It is contemplated that greater or lesser amounts of siRNA can be administered.

Another method for the inhibition of gene expression is based on the use of shorter antisense oligomers consisting of DNA, or other synthetic structural types such as phosphorothiates, 2′-0-alkylribonucleotide chimeras, locked nucleic acid (LNA), peptide nucleic acid (PNA), or morpholinos. With the exception of RNA oligomers, PNAs and morpholinos, all other antisense oligomers act in eukaryotic cells through the mechanism of RNase H-mediated target cleavage. PNAs and morpholinos bind complementary DNA and RNA targets with high affinity and specificity, and thus act through a simple steric blockade of the RNA translational machinery, and appear to be completely resistant to nuclease attack.

Recently it has been shown that morpholino antisense oligonucleotides in zebrafish and frogs overcome the limitations of RNase H-competent antisense oligonucleotides, which include numerous non-specific effects due to the non-target-specific cleavage of other mRNA molecules caused by the low stringency requirements of RNase H. Morpholino oligomers therefore represent an important new class of antisense molecule. Oligomers of the invention may be synthesized by standard methods known in the art. As examples, phosphorothioate oligomers may be synthesized by the method of Stein et al. (1988) Nucleic Acids Res. 16, 3209 3021), methylphosphonate oligomers can be prepared by use of controlled pore glass polymer supports (Sarin et al. (1988) Proc. Natl. Acad. Sci. USA. 85, 7448-7451). Morpholino oligomers may be synthesized by the method of Summerton and Weller U.S. Pat. Nos. 5,217,866 and 5,185,444.

Another particularly form of antisense RNA strategy are gapmers. A gapmer is a chimeric antisense oligonucleotide that contains a central block of deoxynucleotide monomers sufficiently long to induce RNase H cleavage. The central block of a gapmer is flanked by blocks of 2′-O modified ribonucleotides or other artificially modified ribonucleotide monomers such as bridged nucleic acids (BNAs) that protect the internal block from nuclease degradation. Gapmers have been used to obtain RNase-H mediated cleavage of target RNAs, while reducing the number of phosphorothioate linkages. Phosphorothioates possess increased resistance to nucleases compared to unmodified DNA. However, they have several disadvantages. These include low binding capacity to complementary nucleic acids and non-specific binding to proteins that cause toxic side-effects limiting their applications. The occurrence of toxic side-effects together with non-specific binding causing off-target effects has stimulated the design of new artificial nucleic acids for the development of modified oligonucleotides that provide efficient and specific antisense activity in vivo without exhibiting toxic side-effects. By recruiting RNase H, gapmers selectively cleave the targeted oligonucleotide strand. The cleavage of this strand initiates an antisense effect. This approach has proven to be a powerful method in the inhibition of gene functions and is emerging as a popular approach for antisense therapeutics. Gapmers are offered commercially, e.g. LNA longRNA GapmeRs by Exiqon, or MOE gapmers by Isis pharmaceuticals. MOE gapmers or “2′MOE gapmers” are an antisense phosphorothioate oligonucleotide of 15-30 nucleotides wherein all of the backbone linkages are modified by adding a sulfur at the non-bridging oxygen (phosphorothioate) and a stretch of at least 10 consecutive nucleotides remain unmodified (deoxy sugars) and the remaining nucleotides contain an O′-methyl O′-ethyl substitution at the 2′ position (MOE).

Next to the use of the inhibitory RNA technology to reduce or inhibit functional expression of GIUD1 gene on the level of the gene product, inhibitors of functional expression of the GLUD1 can also act at the DNA level. If inhibition is to be achieved at the DNA level, this may be done using gene therapy to knock-out or disrupt the target gene. As used herein, a “knock-out” can be a gene knockdown or the gene can be knocked out by a mutation such as, a point mutation, an insertion, a deletion, a frameshift, or a missense mutation by techniques known in the art, including, but not limited to, retroviral gene transfer. Another way in which genes can be knocked out is by the use of zinc finger nucleases. Zinc-finger nucleases (ZFNs) are artificial restriction enzymes generated by fusing a zinc finger DNA-binding domain to a DNA-cleavage domain. Zinc finger domains can be engineered to target desired DNA sequences, which enable zinc-finger nucleases to target unique sequence within a complex genome. By taking advantage of endogenous DNA repair machinery, these reagents can be used to precisely alter the genomes of higher organisms. Other technologies for genome customization that can be used to knock out genes are meganucleases and TAL effector nucleases (TALENs, Cellectis bioresearch). A TALEN® is composed of a TALE DNA binding domain for sequence-specific recognition fused to the catalytic domain of an endonuclease that introduces double strand breaks (DSB). The DNA binding domain of a TALEN® is capable of targeting with high precision a large recognition site (for instance 17 bp). Meganucleases are sequence-specific endonucleases, naturally occurring “DNA scissors”, originating from a variety of single-celled organisms such as bacteria, yeast, algae and some plant organelles. Meganucleases have long recognition sites of between 12 and 30 base pairs. The recognition site of natural meganucleases can be modified in order to target native genomic DNA sequences (such as endogenous genes). Another recent genome editing technology is the CRISPR/Cas system, which can be used to achieve RNA-guided genome engineering. CRISPR interference is a genetic technique which allows for sequence-specific control of gene expression in prokaryotic and eukaryotic cells. It is based on the bacterial immune system-derived CRISPR (clustered regularly interspaced palindromic repeats) pathway.

Thus, the term “RNA based inhibitor” as used herein refers to an RNA molecule that inhibits the functional expression of the GLUD1 gene. Alternative wording for “RNA based inhibitor” is “inhibitor of the inhibitory RNA technology”. Non-limiting examples of RNA based inhibitors are gapmers, shRNAs, siRNAs, artificial microRNAs, dsRNAs, anti-sense RNA oligomers. “Nucleotide or nucleic acid based inhibitors” as used herein refers to nucleic acid molecules that inhibit the functional expression of the GLUD1 gene. Nucleotide/nucleic acid based inhibitors can be RNA based inhibitors but also DNA based inhibitors. Non-liming example of DNA based inhibitors are anti-sense DNA oligomers, morpholinos, locked nucleic acids, a peptide nucleic acid. The term “inhibitor” as used in this application is a molecule that inhibits the functional expression of the GLUD1 gene or a small compound inhibiting the enzymatic activity (or the functional activity) of the GLUD1 protein. Among nucleotide inhibitors, the term “inhibitor” from this application also envisages non-nucleotide inhibitors. Non-limiting examples of said non-nucleotide inhibitors are ribozymes, peptide nucleic acids, Zinc-finger nucleases, TALENs, CRISPR-Cas, CRISPR-C2c2 or a meganucleases.

Human GLUD1 specific siRNA oligo duplexes, shRNA plasmid kits and shRNA lentiviral particles are commercially available (e.g. Origene Technologies Inc) or can nowadays be easily designed and manufactured. Multiple human GLUD1-specific guide RNAs (gRNAs) are likewise commercially available for use in CRISPR-Cas9 technology (e.g. Genscript, for methodology of designing gRNAs referring to Sanjana et al. 2014, Nat Methods 11:783-784). miRNAs targeting human GLUD1 can be found in the miRTarBase (http://mirtarbase.mbc.nctu.edu.tw/php/index.php) or can nowadays be easily designed and manufactured.

As already hinted at hereinabove, adoptive cell transfer (ACT), in particular adoptive transfer of macrophages can be applied. One type of ACT gaining traction and regulatory approval relies on chimeric antigen receptors (CAR) redirected T cells. As explained in Example 7, adoptive transfer of macrophages has been performed successfully. For purposes of the current invention, isolated macrophages (autologous or allogeneic) are redirected towards GLUD1 inhibition by means of GLUD1 gene knock-out or by means of transient or conditional expression of a GLUD1 inhibitor. The conditional expression can for instance be inducible expression of the GLUD1 inhibitor, or constitutive expression of the GLUD1 inhibitor in conjunction with e.g. inducible expression of a cytotoxic compound or suicide product which would allow for selective elimination of the engineered macrophages in case this would be desired. See also Example 7.

Many different vectors have been used in human nucleic acid therapy trials and a listing can be found on http://www.abedia.com/wiley/vectors.php. Currently the major groups are adenovirus or adeno-associated virus vectors (in about 21% and 7% of the clinical trials), retrovirus vectors (about 19% of clinical trials), naked or plasmid DNA (about 17% of clinical trials), and lentivirus vectors (about 6% of clinical trials). Combinations are also possible, e.g. naked or plasmid DNA combined with adenovirus, or RNA combined with naked or plasmid DNA to list just a few. Other viruses (e.g. alphaviruses) are used in nucleic acid therapy and are not excluded in the context of the current invention.

Administration may be aided by specific formulation of the nucleic acid e.g. in liposomes (lipoplexes) or polymersomes (synthetic variants of liposomes), as polyplexes (nucleic acid complexed with polymers), carried on dendrimers, in inorganic (nano)particles (e.g. containing iron oxide in case of magnetofection), or combined with a cell penetrating peptide (CPP) to increase cellular uptake. Organ- or cellular-targeting strategies may also be applied to the nucleic acid (nucleic acid combined with organ- or cell-targeting moiety); these include passive targeting (mostly achieved by adapted formulation) or active targeting (e.g. by coupling a nucleic acid-comprising nanoparticle with any compound (e.g. an aptamer or antibody or antigen binding molecule) binding to a target organ- or cell-specific antigen) (e.g. Steichen et al. 2013, Eur J Pharm Sci 48:416-427).

CPPs enable translocation of the drug of interest coupled to them across the plasma membrane. CPPs are alternatively termed Protein Transduction Domains (TPDs), usually comprise 30 or less (e.g. 5 to 30, or 5 to 20) amino acids, and usually are rich in basic residues, and are derived from naturally occurring CPPs (usually longer than 20 amino acids), or are the result of modelling or design. A non-limiting selection of CPPs includes the TAT peptide (derived from HIV-1 Tat protein), penetratin (derived from Drosophila Antennapedia—Antp), pVEC (derived from murine vascular endothelial cadherin), signal-sequence based peptides or membrane translocating sequences, model amphipathic peptide (MAP), transportan, MPG, polyarginines; more information on these peptides can be found in Torchilin 2008 (Adv Drug Deliv Rev 60:548-558) and references cited therein. CPPs can be coupled to carriers such as nanoparticles, liposomes, micelles, or generally any hydrophobic particle. Coupling can be by absorption or chemical bonding, such as via a spacer between the CPP and the carrier. To increase target specificity an antibody binding to a target-specific antigen can further be coupled to the carrier (Torchilin 2008, Adv Drug Deliv Rev 60:548-558). CPPs have already been used to deliver payloads as diverse as plasmid DNA, oligonucleotides, siRNA, peptide nucleic acids (PNA), proteins and peptides, small molecules and nanoparticles inside the cell (Stalmans et al. 2013, PloS One 8:e71752).

Targeting mitochondria has also become feasible, e.g. by means of nanoparticles (Wen et al. 2016, Adv Drug Deliv Rev 99A:52-69), or, for e.g. RNA, by means of coupling to an RNA import component (polynucleotide phosphorylase, PNPASE) (Wang et al. 2012, Proc Natl Acad Sci 109:4840-4845).

Any other modification of the DNA or RNA to enhance efficacy of nucleic acid therapy is likewise envisaged to be useful in the context of the applications of the nucleic acid or nucleic acid comprising compound as outlined herein. The enhanced efficacy can reside in enhanced expression, enhanced delivery properties, enhanced stability and the like. The applications of the nucleic acid or nucleic acid comprising compound as outlined herein may thus rely on using a modified nucleic acid as described above. Further modifications of the nucleic acid may include those suppressing inflammatory responses (hypoinflammatory nucleic acids).

Downregulating of expression of a gene encoding a target is feasible through gene therapy (e.g., by administering siRNA, shRNA or antisense oligonucleotides to the target gene). Biopharmaceutical and gene therapeutic antagonists include such entities as antisense oligonucleotides, gapmers, siRNA, shRNA, zinc-finger nucleases, meganucleases, TAL effector nucleases, CRISPR-Cas effectors, antibodies or fragments thereof, alpha-bodies, nanobodies, intrabodies, aptamers, DARPins, affibodies, affitins, anticalins, and monobodies (general description of these compounds included hereinafter).

Inactivation of a process as envisaged in the current invention refers to different possible levels of inactivation, e.g., at least 10%, at least 20%, at least 30%, at least 40%, at least 50%, at least 60%, at least 70%, at least 80%, at least 90%, at least 95%, or even 100% or more of inactivation (compared to a normal situation). The nature of the inactivating compound is not vital/essential to the invention as long as the herein intended therapeutic or prophylactic effect is reached.

An “antagonist” or “inhibitor” or “inactivating compound” refers to a molecule that decreases, blocks, inhibits, abrogates, or interferes with target (i.e. GLUD1) expression, activation or function. In a particular embodiment for an (ant)agonist being a pharmaceutical or biopharmaceutical compound, an (ant)agonist has a binding affinity (dissociation constant) to its target of about 100 μM or less, a binding affinity or about 50 μM or less, a binding affinity of about 10 μM or less, a binding affinity of about 1000 nM or less, a binding affinity to target of about 100 nM or less, a binding affinity to target of about 50 nM or less, a binding affinity to target, of about 10 nM or less, or a binding affinity to target of about 1 nM or less.

In a particular embodiment, an antagonist inhibits target signaling or function with an IC50 of 100 μM or less, with an IC50 of 50 μM or less, with an IC50 of 10 μM or less, with an IC50 of 1000 nM or less, with an IC50 of 500 nM or less, with an IC50 of 100 nM or less, with an IC50 of 50 nM or less, with an IC50 of 10 nM or less, or with an IC50 of 1 nM or less.

In another embodiment any of the GLUD1 inhibitors described above, including GLUD1 knock-out macrophages or macrophages conditionally expressing a GLUD1 inhibitor, are provided for use as a medicament. More precisely, an inhibitor of functional expression of GLUD1 or an inhibitor of the functional activity of GLUD1 is provided for use as a medicament. In a particular embodiment, said inhibitor is a nucleotide based inhibitor. In another particular embodiment, said inhibitor is an RNA based inhibitor. In yet another embodiment said inhibitor is a small compound acting on the GLUD1 protein and inhibiting its enzymatic activity. In other particular embodiments, an inhibitor or nucleotide based inhibitor or an RNA based inhibitor of the functional expression of GLUD1 is provided for use as a medicament, wherein said inhibitor is a GLUD1-selective nucleic acid based inhibitor selected from a gapmer, a shRNA, a siRNA, an artificial microRNA, a dsRNA, an anti-sense oligomer, a ribozyme, a morpholino, a locked nucleic acid, a peptide nucleic acid, a Zinc-finger nuclease, a TALEN, a CRISPR-Cas, a CRISPR-C2c2, a meganuclease or a small compound inhibiting the activity of GLUD1.

In yet another embodiment the invention provides pharmaceutical compositions comprising an inhibitor of GLUD1 as herein before described, including GLUD1 knock-out macrophages or macrophages conditionally expressing a GLUD1 inhibitor, for the treatment of muscle wasting diseases, in particular for use in any of the aspects and embodiments described hereinabove. In particular, the pharmaceutical composition comprising a GLUD1-inhibitor is further comprising an excipient. An excipient can be included for purpose of long-term stabilization, bulking up solid formulations that contain potent active ingredients in small amounts (in that case sometimes referred to as bulking agents or fillers or diluents), or to confer a therapeutic enhancement on the active ingredient in the final dosage form, such as facilitating absorption of the active pharmaceutical ingredient, reducing its viscosity, or enhancing its solubility. An excipient in general may enhance the pharmacokinetic/pharmacodynamics properties of the active pharmaceutical ingredient.

The pharmaceutical compositions of this application may be in the form of oil-in-water emulsions. The emulsions may also contain sweetening and flavoring agents. Oily suspensions may be formulated by suspending the active ingredient in a vegetable oil such as, for example, arachis oil, olive oil, sesame oil or coconut oil, or in a mineral oil such as liquid paraffin. The pharmaceutical compositions may be in the form of sterile injectable aqueous suspensions. Such suspensions may be formulated according to known methods using suitable dispersing or wetting agents and suspending agents, all well-known by the person skilled in the art. The sterile injectable preparation may also be a sterile injectable solution or suspension in a non-toxic parenterally acceptable diluent or solvent. Diluents and solvents that may be employed are, for example, water, Ringer's solution, isotonic sodium chloride solutions and isotonic glucose solutions. In addition, sterile fixed oils are conventionally employed as solvents or suspending media. For this purpose, any bland, fixed oil may be employed including synthetic mono- or diglycerides. In addition, fatty acids such as oleic acid can be used in the preparation of injectables. The compositions of the invention can also contain other conventional pharmaceutically acceptable compounding ingredients, generally referred to as carriers or diluents, as necessary or desired. The nature of additional ingredients and the need of adding those to the composition of the invention is within the knowledge of a skilled person in the relevant art. Conventional procedures for preparing such compositions in appropriate dosage forms can be utilized. Such ingredients and procedures include those described in the following references, each of which is incorporated herein by reference: Powell, M. F. et al., “Compendium of Excipients for Parenteral Formulations” PDA Journal of Pharmaceutical Science & Technology 1998, 52(5), 238-311; Strickley, R. G “Parenteral Formulations of Small Molecule Therapeutics Marketed in the United States (1999)—Part-1” PDA Journal of Pharmaceutical Science & Technology 1999, 53(6), 324-349; and Nema, S. et al., “Excipients and Their Use in Injectable Products” PDA Journal of Pharmaceutical Science & Technology 1997, 51 (4), 166-171.

In a specific embodiment delivery to an affected limb (e.g. where muscle wasting or muscle loss has occurred) is preferred. For local delivery to the muscle or to the affected limb, the pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. The clinician can choose a sustained release or longer acting formulation. Thus, the procedure can be repeated only every several months, depending on the subject's tolerance of the treatment and response.

The present invention not only aims at using inhibitors of GLUD1 for treatment of humans but also aims at using these molecules for veterinary diseases and conditions. Common causes of myopathies (degenerative diseases of muscle) in animals are: (1) metabolic myopathies (e.g. porcine stress syndrome, malignant hyperthermia and pale soft exudative pork), (2) exertional myopathies which comprise a group of diseases which result in severe muscle degeneration following strenuous exercise (e.g. azoturia and tying-up in horses, greyhound myopathy in dogs, capture myopathy in wild animals and compartment syndrome in poultry), (3) traumatic myopathies (e.g. Downer syndrome which is an ischemic necrosis of ventral and limb muscles following prolonged recumbency (disease/anesthesia) and Crush syndrome). As such, when herein referring to a “subject”, humans and animals are meant. In particular the subject may be a mammalian subject.

### Treatment/Therapeutically Effective Amount

“Treatment”/“treating” refers to any rate of reduction, delaying or retardation of the progress of the disease or disorder, or a single symptom thereof, compared to the progress or expected progress of the disease or disorder, or singe symptom thereof, when left untreated. This implies that a therapeutic modality on its own may not result in a complete or partial response (or may even not result in any response), but may, in particular when combined with other therapeutic modalities, contribute to a complete or partial response (e.g. by rendering the disease or disorder more sensitive to therapy). More desirable, the treatment results in no/zero progress of the disease or disorder, or singe symptom thereof (i.e. “inhibition” or “inhibition of progression”), or even in any rate of regression of the already developed disease or disorder, or singe symptom thereof. “Suppression/suppressing” can in this context be used as alternative for “treatment/treating”. Treatment/treating also refers to achieving a significant amelioration of one or more clinical symptoms associated with a disease or disorder, or of any single symptom thereof. Depending on the situation, the significant amelioration may be scored quantitatively or qualitatively. Qualitative criteria may e.g. by patient well-being. In the case of quantitative evaluation, the significant amelioration is typically a 10% or more, a 20% or more, a 25% or more, a 30% or more, a 40% or more, a 50% or more, a 60% or more, a 70% or more, a 75% or more, a 80% or more, a 95% or more, or a 100% improvement over the situation prior to treatment. The time-frame over which the improvement is evaluated will depend on the type of criteria/disease observed and can be determined by the person skilled in the art.

A “therapeutically effective amount” refers to an amount of a therapeutic agent to treat or prevent a disease, disorder, or unwanted condition in a subject. The term “effective amount” refers to the dosing regimen of the agent (e.g. antagonist as described herein) or composition comprising the agent (e.g. medicament or pharmaceutical composition). The effective amount will generally depend on and/or will need adjustment to the mode of contacting or administration. The effective amount of the agent or composition comprising the agent is the amount required to obtain the desired clinical outcome or therapeutic effect without causing significant or unnecessary toxic effects (often expressed as maximum tolerable dose, MTD). To obtain or maintain the effective amount, the agent or composition comprising the agent may be administered as a single dose or in multiple doses (see explanation on single administrations), such as to obtain or maintain the effective amount over the desired time span/treatment duration. The effective amount may further vary depending on the severity of the condition that needs to be treated; this may depend on the overall health and physical condition of the mammal or patient and usually the treating doctor's or physician's assessment will be required to establish what is the effective amount. The effective amount may further be obtained by a combination of different types of contacting or administration.

The aspects and embodiments described above in general may comprise the administration of one or more therapeutic compounds to a subject in need thereof, i.e., in need of treatment. In general a (therapeutically) effective amount of (a) therapeutic compound(s) is administered to the subject in need thereof in order to obtain the described clinical response(s). “Administering” means any mode of contacting that results in interaction between an agent (e.g. a therapeutic compound) or composition comprising the agent (such as a medicament or pharmaceutical composition) and an object (e.g. cell, tissue, organ, body lumen) with which said agent or composition is contacted. The interaction between the agent or composition and the object can occur starting immediately or nearly immediately with the administration of the agent or composition, can occur over an extended time period (starting immediately or nearly immediately with the administration of the agent or composition), or can be delayed relative to the time of administration of the agent or composition. More specifically the “contacting” results in delivering an effective amount of the agent or composition comprising the agent to the object.

A single administration of a pharmacologic compound in general leads to a transient effect due to its gradual removal from the cell, organ and/or body and is reflected in the pharmacokinetic/-dynamic behavior of the compound. Depending on the desired level of GLUD1 inactivation, two or more (multiple) administrations of the pharmacologic compound may be required. Inactivation by gene or nucleic acid therapy or by a gene therapeutic compound (nucleic acid or nucleic acid comprising compound) can be inducible when controlled by a promoter responsive to a to be administered signal not normally present in the target cell, -organ, or -body. As such, the inactivation by gene or nucleic acid therapy may be transient (e.g. upon removal or disappearance of the administered signal from the target cell, -organ, or -body). In case of a nucleic acid or nucleic acid comprising compound degrading once inside the target cell, -organ, or -body (e.g. in case when not integrated in the genome), the effect of the compound generally is transient.

### Other Definitions

The present invention will be described with respect to particular embodiments and with reference to certain drawings but the invention is not limited thereto but only by the claims. Any reference signs in the claims shall not be construed as limiting the scope. The drawings described are only schematic and are non-limiting. In the drawings, the size of some of the elements may be exaggerated and not drawn on scale for illustrative purposes. Where the term “comprising” is used in the present description and claims, it does not exclude other elements or steps. Where an indefinite or definite article is used when referring to a singular noun e.g. “a” or “an”, “the”, this includes a plural of that noun unless something else is specifically stated. Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein. T

Terms or definitions described hereinabove and hereunder are provided solely to aid in the understanding of the invention. Unless specifically defined herein, all terms used herein have the same meaning as they would to one skilled in the art of the present invention. In relation to molecular biology, practitioners are particularly directed to Sambrook et al., Molecular Cloning: A Laboratory Manual, 4th ed., Cold Spring Harbor Press, Plainsview, N.Y. (2012); and Ausubel et al., current Protocols in Molecular Biology (Supplement 100), John Wiley & Sons, New York (2012), for definitions and terms of the art. None of the definitions provided herein should not be construed to have a scope less than understood by a person of ordinary skill in the art.

The term “defined by SEQ ID NO:X” as used herein refers to a biological sequence consisting of the sequence of amino acids or nucleotides given in the SEQ ID NO:X. For instance, an antigen defined in/by SEQ ID NO:X consists of the amino acid sequence given in SEQ ID NO:X. A further example is an amino acid sequence comprising SEQ ID NO:X, which refers to an amino acid sequence longer than the amino acid sequence given in SEQ ID NO:X but entirely comprising the amino acid sequence given in SEQ ID NO:X (wherein the amino acid sequence given in SEQ ID NO:X can be located N-terminally or C-terminally in the longer amino acid sequence, or can be embedded in the longer amino acid sequence), or to an amino acid sequence consisting of the amino acid sequence given in SEQ ID NO:X. The description of the genes as included hereinabove refer to “reference mRNA sequences”, these can be found be searching e.g. GenBank. The listed reference mRNA sequences all refer to human sequences and neither of the listings is meant to be exhaustive. Based on the listed sequences, a skilled person will be able to retrieve e.g. genomic sequences, other mRNA sequences and encoded protein sequences either of human or other mammalian origin (e.g. by applying the BLAST tool publicly available via e.g. NCBI).

### Chemical Definitions

As used herein, “alkyl” means a noncyclic straight chain or branched, unsaturated or saturated hydrocarbon such as those containing from 1 to 10 carbon atoms (C1-C10, or C₁₋₁₀). A “higher alkyl” refers to unsaturated or saturated hydrocarbon having 6 or more carbon atoms. Exemplary saturated straight chain alkyls include methyl, ethyl, n-propyl, n-butyl, n-pentyl, n-hexyl, n-septyl, n-octyl, n-nonyl, and the like; exemplary saturated branched alkyls include isopropyl, sec-butyl, isobutyl, tert-butyl, isopentyl, and the like. Unsaturated alkyls comprise at least one double or triple bond between adjacent carbon atoms (referred to as an “alkenyl” or “alkynyl”, respectively). Exemplary straight chain and branched alkenyls include ethylenyl, propylenyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and the like; exemplary straight chain and branched alkynyls include acetylenyl, propynyl, 1-butynyl, 2-butynyl, 1-pentynyl, 2-pentynyl, 3-methyl-1-butynyl, and the like.

Non-aromatic mono or polycyclic alkyls are referred to herein as “carbocycles” or “carbocyclyl” groups. Examples of saturated carbocycles include cyclopropyl, cyclobutyl, cyclopentyl, cyclohexyl, and the like; while unsaturated carbocycles include cyclopentenyl and cyclohexenyl, and the like.

“Heterocarbocycles” or heterocarbocyclyl” groups are carbocycles which contain from 1 to 4 heteroatoms independently selected from nitrogen, oxygen and sulphur which may be saturated or unsaturated (but not aromatic), monocyclic or polycyclic, and wherein the nitrogen and sulphur heteroatoms may be optionally oxidized, and the nitrogen heteroatom may be optionally quaternized. Heterocarbocycles include morpholinyl, pyrrolidinonyl, pyrrolidinyl, piperidinyl, hydantoinyl, valerolactamyl, oxiranyl, oxetanyl, tetrahydrofuranyl, tetrahydropyranyl, tetrahydropyridinyl, tetrahydroprimidinyl, tetrahydrothiophenyl, tetrahydrothiopyranyl, tetrahydropyrimidinyl, tetrahydrothiophenyl, tetrahydrothiopyranyl, and the like.

The term “aryl” refers to aromatic homocyclic (i.e., hydrocarbon) mono-, bi- or tricyclic ring-containing groups such as having 6 to 12 members such as phenyl, naphthyl and biphenyl.

As used herein, “heteroaryl” or “heteroaromatic” refers an aromatic heterocarbocycle having 1 to 4 heteroatoms selected from nitrogen, oxygen and sulfur, and containing at least 1 carbon atom, including both mono- and polycyclic ring systems. Polycyclic ring systems may, but are not required to, contain one or more non-aromatic rings, as long as one of the rings is aromatic. Examples of heteroaryls include furyl, benzofuranyl, thiophenyl, benzothiophenyl, pyrrolyl, indolyl, isoindolyl, azaindolyl, pyridyl, quinolinyl, isoquinolinyl, oxazolyl, isooxazolyl, benzoxazolyl, pyrazolyl, imidazolyl, benzimidazolyl, thiazolyl, benzothiazolyl, isothiazolyl, pyridazinyl, pyrimidinyl, pyrazinyl, triazinyl, cinnolinyl, phthalazinyl, and quinazolinyl. “Heteroaryls” includes N-alkylated derivatives such as a 1-methylimidazol-5-yl substituents.

As used herein, “heterocycle” or “heterocyclyl” refers to mono- and polycyclic ring systems having 1 to 4 heteroatoms selected from nitrogen, oxygen and sulfur, and containing at least 1 carbon atom. The mono- and polycyclic ring systems may be aromatic, non-aromatic or mixtures of aromatic and non-aromatic rings. Heterocycles includes heterocarbocycles, heteroaryls, and the like.

“Alkoxy” refers to an alkyl group as defined above with the indicated number of carbon atoms attached through an oxygen bridge. Examples of alkoxy compounds include, but are not limited to, methoxy, ethoxy, n-propoxy, i-propoxy, n-butoxy, s-butoxy, t-butoxy, n-pentoxy, and s-pentoxy. Related to alkoxy groups are aryloxy groups, which have an aryl group singular bonded to oxygen such as the phenoxy group (C₆H₅O—).

“Alkoxyalkyl” refers an alkyl group as defined above with the indicated number of carbon atoms attached through an alkyl bridge (i.e., —CH2-O—CH2CH3).

An alkoxy or aryloxy group bonded to an alkyl or aryl (R¹—O—R²) is an ether. If bonded to H it is an alcohol. An alkoxide (RO⁻) is the ionic or salt form; it is a derivative of an alcohol where the proton has been replaced by a e.g. a metal ion such as a sodium ion.

“Alkylamino” refers an alkyl group as defined above with the indicated number of carbon atoms attached through an amino bridge. An example of an alkylamino is methylamino, (i.e., —NH—CH3).

“Alkylthio” refers to an alkyl group as defined above with the indicated number of carbon atoms attached through a sulfur bridge. An example of an alkylthio is methylthio, (i.e., —S—CH3).

“Alkanoyl” refers to an alkyl as defined above with the indicated number of carbon atoms attached through a carbonyl bride (i.e., —(C═O)alkyl).

The terms “cycloalkyl” and “cycloalkenyl” refer to mono-, bi-, or tri homocyclic ring groups of 3 to 15 carbon atoms which are, respectively, fully saturated and partially unsaturated.

The terms “halogen” or “Hal” refer to fluorine (F), chlorine (Cl), bromine (Br), and iodine (I). The term “halogenation” in general refers to a halogen substituent substituting a hydrogen in a compound.

The term “substituted” refers to a molecule wherein at least one hydrogen atom is replaced with a substituent. When substituted, one or more of the groups are “substituents.” The molecule may be multiply substituted. Substitution may be optional, meaning that it is possible for the designated atom to be unsubstituted. In the case of an oxo substituent (“═O”), two hydrogen atoms are replaced. Examplary substituents include halogen, hydroxy, alkyl, alkoxy, nitro, cyano, oxo, carbocyclyl, carbocycloalkyl, heterocarbocyclyl, heterocarbocycloalkyl, aryl, a ryloxy, a rylalkyl, heteroaryl, heteroarylalkyl, —NRaRb, —NRaC(═O)Rb, —NRaC(═O)NRaNRb, —NRaC(═O)ORb, —NRaSO2Rb, —C(═O)Ra, —C(═O)ORa, —C(═O)NRaRb, —OC(═O)NRaRb, —ORa, —SRa, —SORa, —S(═O)2Ra, —OS(═O)2Ra and —S(═O)2ORa. Ra and Rb in this context may be the same or different and independently hydrogen, halogen hydroxyl, alkyl, alkoxy, alkyl, amino, alkylamino, dialkylamino, carbocyclyl, carbocycloalkyl, heterocarbocyclyl, heterocarbocycloalkyl, aryl, a ryloxy, a rylalkyl, heteroaryl, heteroarylalkyl.

As used herein, the term “derivative” refers to a structurally similar compound that retains sufficient functional attributes of the identified analogue. The derivative may be structurally similar because it is lacking one or more atoms, substituted, a salt, in different hydration/oxidation states, or because one or more atoms within the molecule are switched, such as, but not limited to, adding a hydroxyl group, replacing an oxygen atom with a sulfur atom, or replacing an amino group with a hydroxyl group, oxidizing a hydroxyl group to a carbonyl group, reducing a carbonyl group to a hydroxyl group, and reducing a carbon-to-carbon double bond to an alkyl group or oxidizing a carbon-to-carbon single bond to a double bond. A derivative optional has one or more, the same or different, substitutions. Derivatives may be prepare by any variety of synthetic methods or appropriate adaptations presented in synthetic or organic chemistry text books, such as those provide in March's Advanced Organic Chemistry: Reactions, Mechanisms, and Structure, Wiley, 6th Edition (2007) Michael B. Smith or Domino Reactions in Organic Synthesis, Wiley (2006) Lutz F. Tietze hereby incorporated by reference.

The term “prodrug” refers to a compound that undergoes biotransformation before exhibiting pharmacological effects. Prodrugs can thus be viewed as drugs containing specialized nontoxic protective groups used in a transient manner to alter or to eliminate undesirable properties in the parent molecule (from: Vert et al. 2012, Pure Appl Chem 84:377-410). The protective groups can have one or more function such as increasing bioavailability, increasing solubility, increasing stability, avoiding or reducing premature release of the drug (thus avoiding or reducing toxicity), supporting administration of the drug to the targeted cells or organs in a subject, etc.

Co-crystals are crystalline materials composed of two or more different molecules, typically an active pharmaceutical ingredient (API) or drug and co-crystal formers (“coformers”), in the same crystal lattice. Pharmaceutical co-crystals have opened up opportunities for engineering solid-state forms beyond conventional solid-state forms of an API or drug, such as salts and polymorphs. Co-crystals are readily distinguished from salts because unlike salts, their components are in a neutral state and interact nonionically. In addition, co-crystals differ from polymorphs, which are defined as including only single-component crystalline forms that have different arrangements or conformations of the molecules in the crystal lattice, amorphous forms, and multicomponent phases such as solvate and hydrate forms. Instead co-crystals are more similar to solvates, in that both contain more than one component in the lattice. From a physical chemistry perspective, co-crystals can be viewed as a special case of solvates and hydrates, wherein the second component, the coformer, is nonvolatile. Therefore, co-crystals are classified as a special case of solvates in which the second component is nonvolatile. Co-crystals: Crystalline materials composed of two or more different molecules within the same crystal lattice that are associated by nonionic and noncovalent bonds. Polymorphs: Different crystalline forms of the same API. This may include solvation or 1 hydration products (also known as pseudopolymorphs) and amorphous forms. Per the current regulatory scheme, different polymorphic forms are considered the same APIs. Salts: Any of numerous compounds that result from replacement of part or all of the acid hydrogen of an acid by a metal or a radical acting like a metal: an ionic or electrovalent crystalline compound. Per the current regulatory scheme, different salt forms of the same active moiety are considered different APIs. (from: FDA draft guidance for industry “Regulatory Classification of Pharmaceutical Co-Crystals”; August 2016).

When herein referring to an “analogue” of a chemical compound A, it is generally meant to refer to a structural analogue or chemical analogue of compound A. Analogues of a compound A include, but are not limited to isomers of that compound A. For purposes of the invention, analogues of a GLUD1 inhibiting compound/small molecule should also be functional analogues in that they should as well be capable of inhibiting GLUD1 activity.

### Isomers

Stereoisomeric molecules, or stereoisomers, contain the same atoms linked together in the same sequence (same molecular formula), but having different three-dimensional organizations or configurations.

Optical isomers, also sometimes referred to as enantiomers, are molecules which are non-superposable mirror images of each other. Depending on the optical activity, enantiomers are often described as left- or right-handed, and each member of the pair is referred to as enantiomorph (each enantiomorph being a molecule of one chirality). Mixtures of equal parts of two enantiomorphs are often referred to as racemic mixtures. Compounds comprising within the limits of detection only one enantiomorph are referred to as enantiopure compounds. Optical isomers can occur when molecules comprise one or more chiral centers.

Geometric isomers usually refer to cis-trans isomers wherein rotation around a chemical bond is impossible. Cis-trans isomers often are found in molecules with double or triple bonds.

Structural isomers contain the same atoms (same molecular formula), but linked together in a different sequence.

### Biopharmaceutical Agents

Interfering with structure, which can result in inhibition of function, can be achieved by e.g. moieties binding to the protein of interest, i.e. binding to GLUD1, and therewith inhibiting, blocking, or neutralizing its activity, in particular its enzymatic activity. Non-limiting examples are (monoclonal) antibodies or antigen-binding fragments thereof, alpha-bodies, nanobodies, intrabodies (antibodies binding and/or acting to intracellular target; this typically requires the expression of the antibody within the target cell, which can be accomplished by gene therapy), aptamers, DARPins, affibodies, affitins, anticalins, monobodies, phosphatases (in case of phosphorylated target) and kinases (in case of a phosphorylatable target).

The term “antibody” as used herein refers to any naturally occurring format of antibody or antigen binding protein the production of which is induced by an immune system (immunoglobulins or IgGs). It is clear, however, that not all antibodies are naturally occurring as e.g. some antigens are problematic in the sense that they are poor or not at all immunogenic, or are not recognized by the immune system (e.g. self-antigens); artificial tricks may be required to obtain antibodies against such antigens (e.g. knock-out mice: e.g. Declercq et al. 1995, J Biol Chem 270:8397-8400; DNA immunization for e.g. transmembrane antigens; e.g. Liu et al. 2016, Emerg Microbes Infect 5:e33). “Conventional” antibodies comprise two heavy chains linked together by disulfide bonds and two light chains, one light chain being linked to each of the heavy chains by disulfide bonds. Each heavy chain has at one end a variable domain (VH) followed by a number of constant domains (three or four constant domains, CH1, CH2, CH3 and CH4, depending on the antibody class). Each light chain has a variable domain (VL) at one end and a constant domain (CL) at its other end; the constant domains of the light chains each align with the first constant domains of the heavy chains, and the light chain variable domains each align with the variable domains of the heavy chains. This type of antibodies exist in camels, dromedaries and llamas along with an “unconventional” naturally occurring type of antibodies consisting of only two heavy chains, and thus being devoid of light chains. Other “unconventional” naturally occurring antibodies exist in in the serum of nurse sharks (Ginglymostomatidae) and wobbegong sharks (Orectolobidae). These latter antibodies are called Ig new antigen receptors (IgNARs). They are disulfide-bonded homodimers consisting of five constant domains (CNAR) and one variable domain (VNAR). There is no light chain, and the individual variable domains are independent in solution and do not appear to associate across a hydrophobic interface (Greenberg et al. 1995, Nature 374:168-173; Nuttall et al. 2001, Mol Immunol 38:313-326; Diaz et al. 2002, Immunogenetics 54:501-512; Nuttall et al. 2003, EurJ Biochem 270:3543-3554). Due to the heavy chain dimer structure characteristic of camelid and shark antibodies, these are sometimes termed “Heavy-Chain Mini-Antibodies” (mnHCAbs) or simply “Mini-Antibodies” (mnAbs) (Holliger & Hudson 2005, Nature Biotechnol 23:1126-1136). Without the light chain, these heavy-chain antibodies bind to their antigens by one single domain, the variable antigen binding domain of the heavy-chain immunoglobulin, referred to as Vab (camelid antibodies) or V-NAR (shark antibodies). These smallest intact and independently functional antigen binding fragment Vab is referred to as nano-antibody or nanobody (Muyldermans 2001, J Biotechnol 74:277-302). Multivalent (etc. divalent, trivalent, tetravalent and pentavalent) Vab and/or V-NAR domains may be preferred in some instances due to their potentially higher cellular intake and retention and may be made by recombinant technology or by chemical means, such as described in WO 2010/033913. The variable domains of the light and/or heavy chains are involved directly in binding the antibody to the antigen. An antibody, or antibody fragment as described hereafter, may also be part of a multivalent and/or multispecific antigen binding molecule. An overview of e.g. available bispecific formats (around 100) is provided in Brinkmann & Kontermann 2017 (mAbs 9:182-212). The term “antibody fragment” refers to any molecule comprising one or more fragments (usually one or more CDRs) of an antibody (the parent antibody) such that it binds to the same antigen to which the parent antibody binds. Antibody fragments include Fv, Fab, Fab′, Fab′-SH, single-chain antibody molecules (such as scFv), F(ab′) 2, single variable VH domains, and single variable VL domains (Holliger & Hudson 2005, Nature Biotechnol 23:1126-1136), Vab and V-NAR. The term further includes microantibodies, i.e. the minimum recognition unit of a parent antibody usually comprising just one CDR (Heap et al. 2005, J Gen Virol 86:1791-1800). Any of the fragments can be incorporated in a multivalent and/or multispecific larger molecule, e.g. mono- or bi-specific Fab 2, mono- or tri-specific Fab 3, bis-scFv (mono- or bispecific), diabodies (mono- or bi-specific), triabodies (e.g. trivalent monospecific), tetrabodies (e.g. tetravalent monospecific), minibodies and the like (Holliger & Hudson 2005, Nature Biotechnol 23:1126-1136). Any of the fragments can further be incorporated in e.g. V-NAR domains of shark antibodies or VhH domains of camelid antibodies (nanobodies). All these are included in the term “antibody fragment”.

Alphabodies are also known as Cell-Penetrating Alphabodies and are small 10 kDa proteins engineered to bind to a variety of antigens.

Aptamers have been selected against small molecules, toxins, peptides, proteins, viruses, bacteria, and even against whole cells. DNA/RNA/XNA aptamers are single stranded and typically around 15-60 nucleotides in length although longer sequences of 220 nt have been selected; they can contain non-natural nucleotides (XNA) as described for antisense RNA. A nucleotide aptamer binding to the vascular endothelial growth factor (VEGF) was approved by FDA for treatment of macular degeneration. Variants of RNA aptamers are spiegelmers are composed entirely of an unnatural L-ribonucleic acid backbone. A Spiegelmer of the same sequence has the same binding properties of the corresponding RNA aptamer, except it binds to the mirror image of its target molecule. Peptide aptamers consist of one (or more) short variable peptide domains, attached at both ends to a protein scaffold, e.g. the Affimer scaffold based on the cystatin protein fold. A further variation is described in e.g. WO 2004/077062 wherein e.g. 2 peptide loops are attached to an organic scaffold. Phage-display screening of such peptides has proven to be possible in e.g. WO 2009/098450.

DARPins stands for designed ankyrin repeat proteins. DARPin libraries with randomized potential target interaction residues, with diversities of over 10″12 variants, have been generated at the DNA level. From these, DARPins can be selected for binding to a target of choice with picomolar affinity and specificity. Affitins, or nanofitins, are artificial proteins structurally derived from the DNA binding protein Sac7d, found in Sulfolobus acidocaldarius. By randomizing the amino acids on the binding surface of Sac7d and 5 subjecting the resulting protein library to rounds of ribosome display, the affinity can be directed towards various targets, such as peptides, proteins, viruses, and bacteria.

Anticalins are derived from human lipocalins which are a family of naturally binding proteins and mutation of amino acids at the binding site allows for changing the affinity and selectivity towards a 10 target of interest. They have better tissue penetration than antibodies and are stable at temperatures up to 70° C.

Monobodies are synthetic binding proteins that are constructed starting from the fibronectin type III domain (FN3) as a molecular scaffold.

In the above, the molecules are specific to their intended target, which is referring to the fact that the molecules are acting at the level of the intended target and not at the level of target different from the intended target. Specificity can be ascertained by e.g. determining physical interaction of the molecules to their intended target.

It is to be understood that although particular embodiments, specific configurations as well as materials and/or molecules, have been discussed herein for cells and methods according to the present invention, various changes or modifications in form and detail may be made without departing from the scope and spirit of this invention. The following examples are provided to better illustrate particular embodiments, and they should not be considered limiting the application. The application is limited only by the claims.

### Examples

### 1. GLUD1 Deficiency in Macrophages Improves the Outcome of Skeletal Muscle Damage

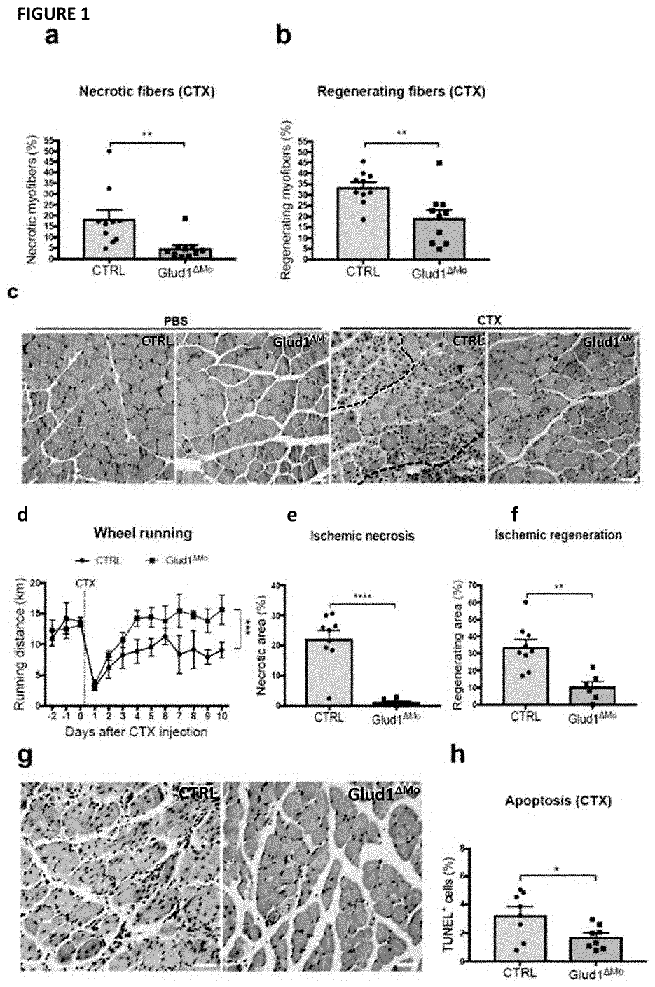

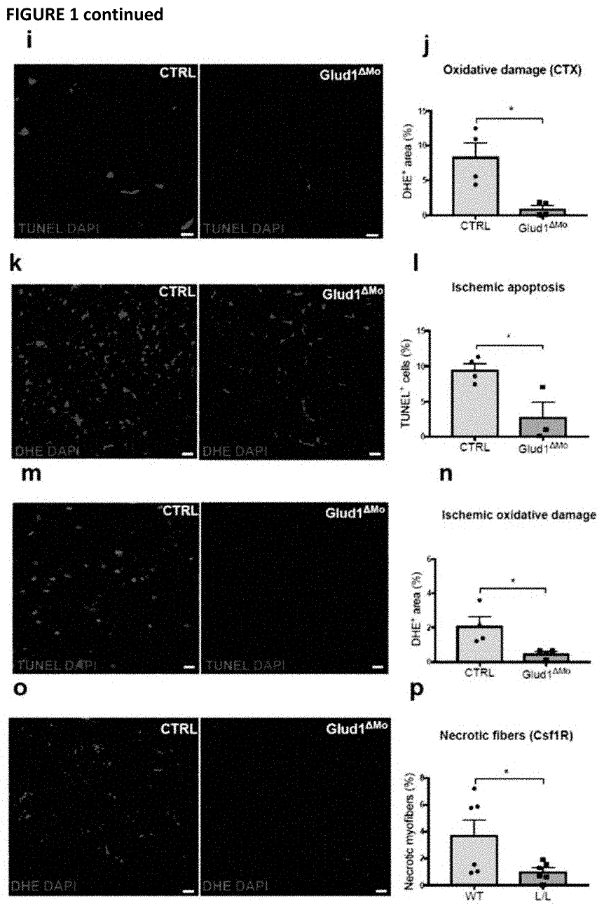

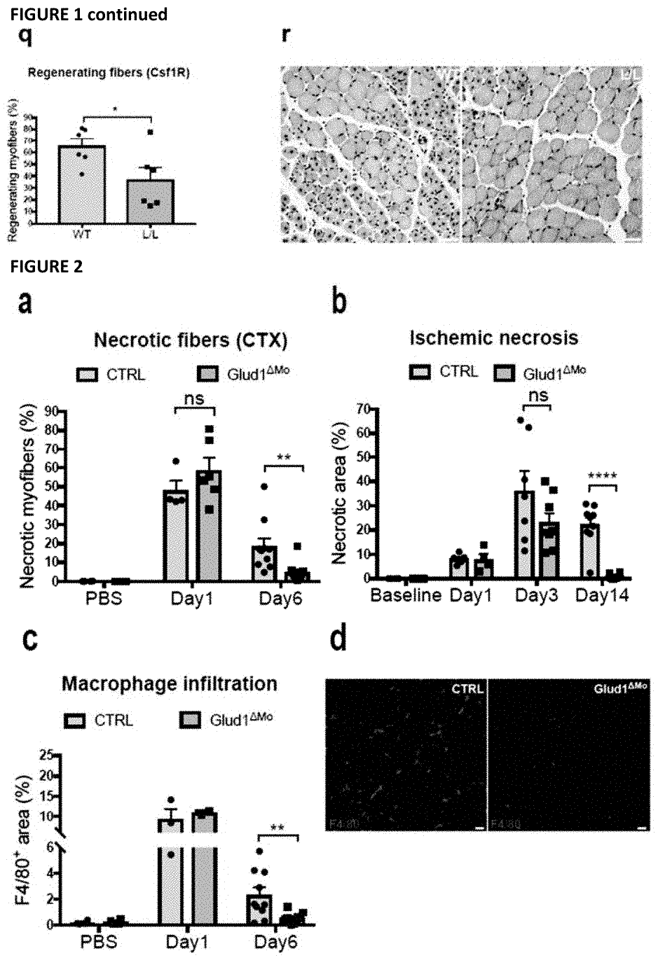

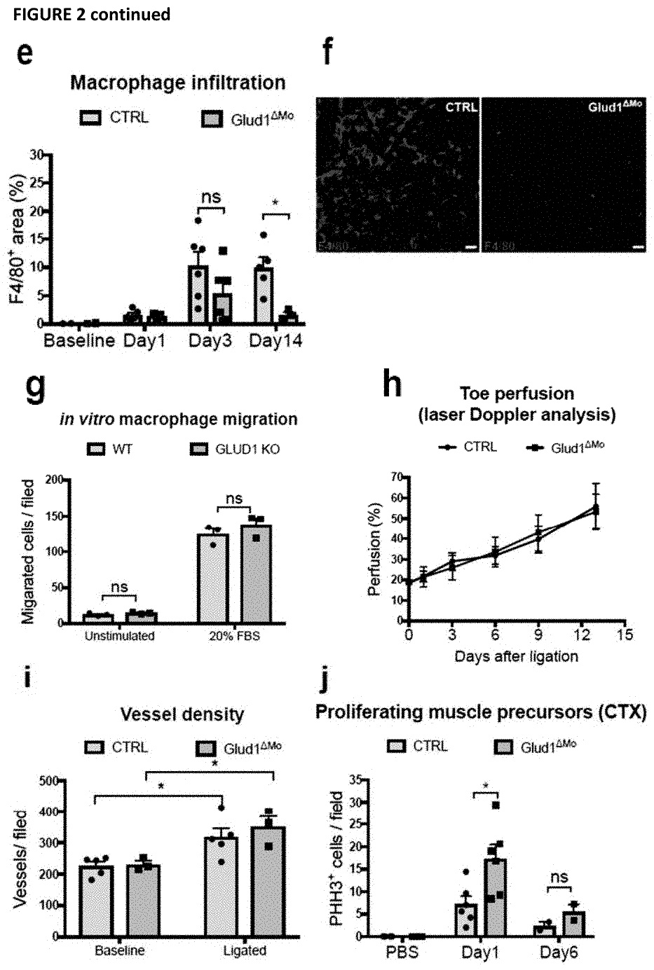

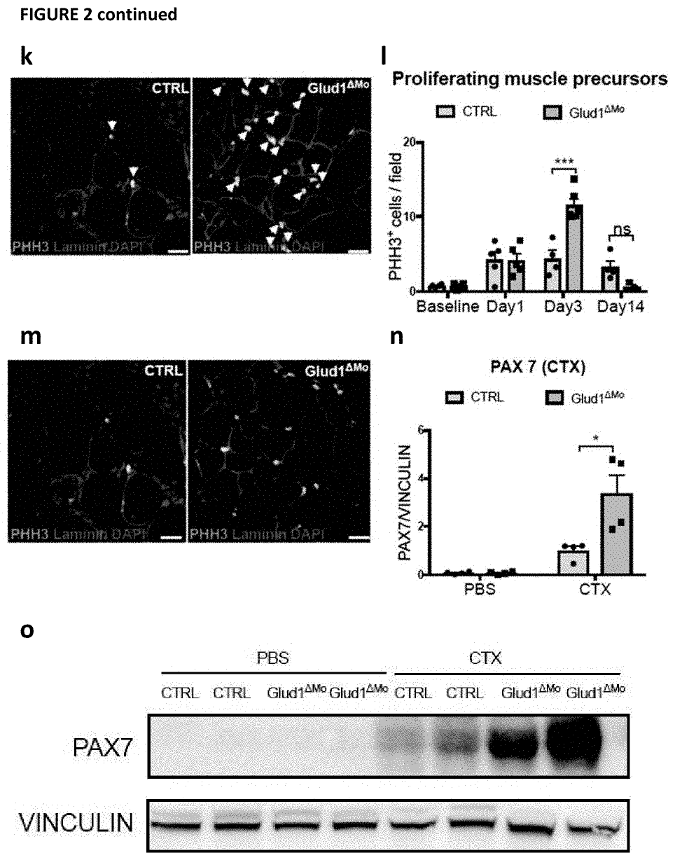

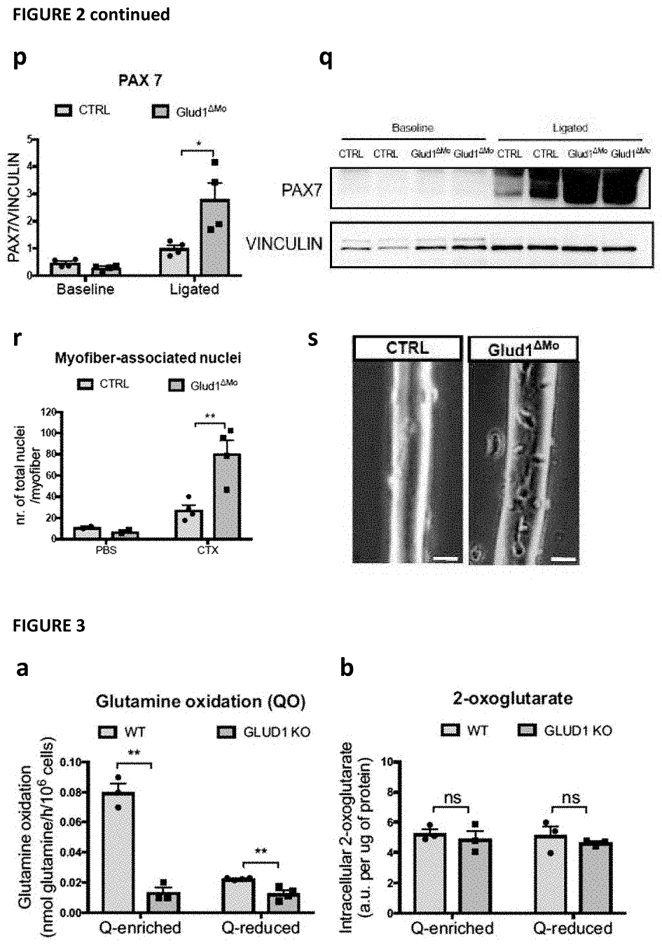

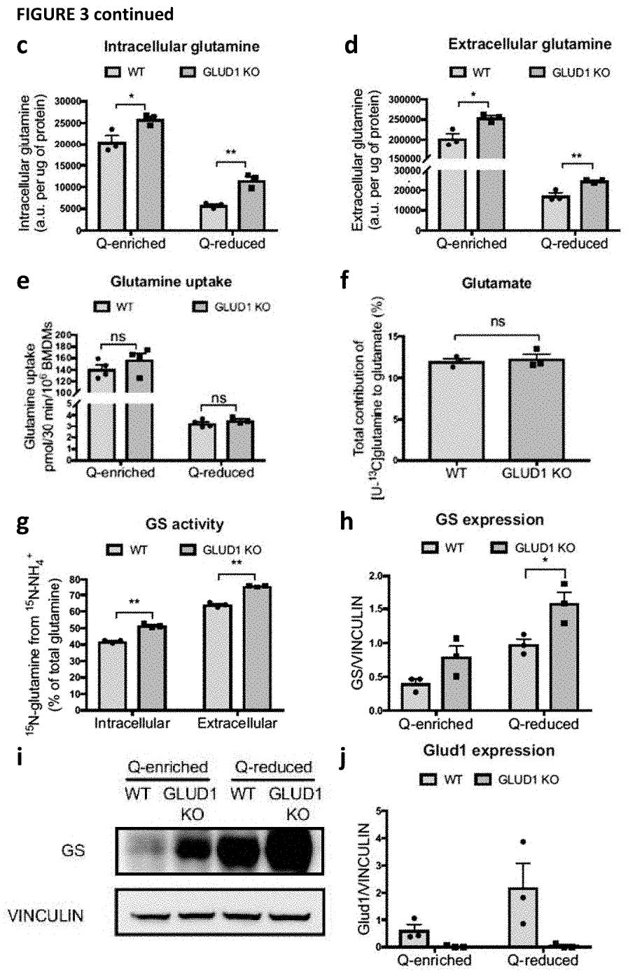

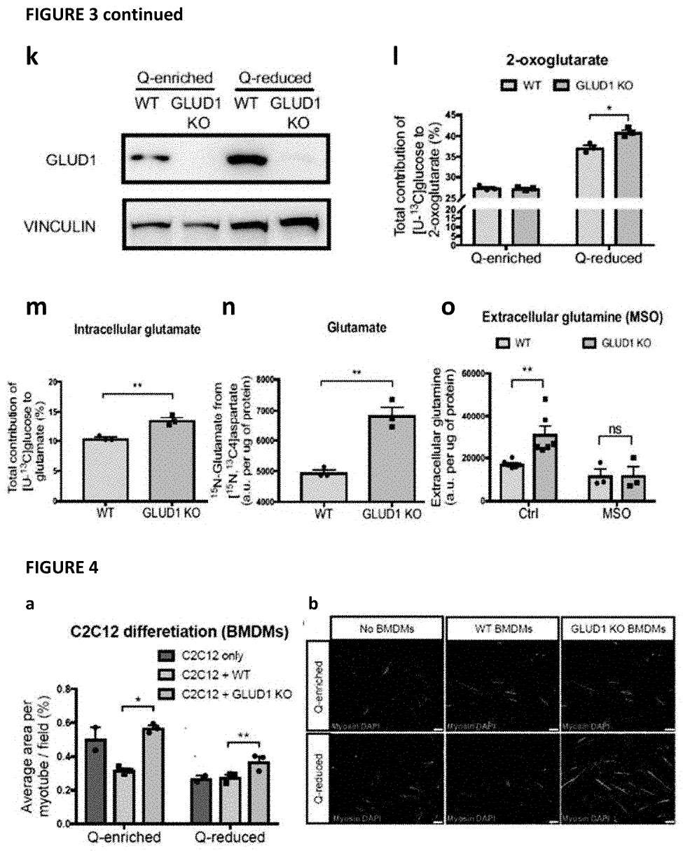

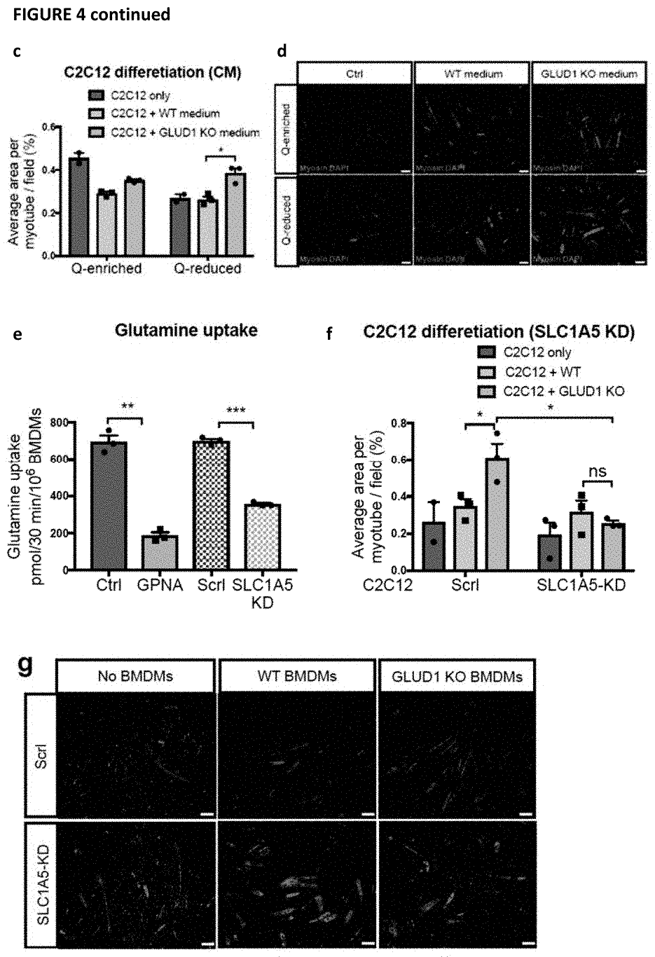

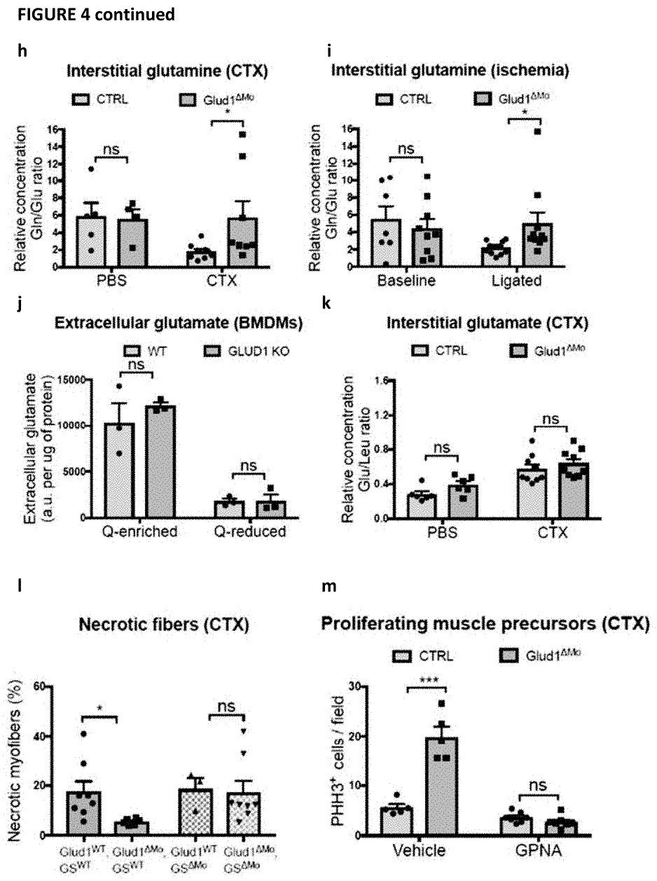

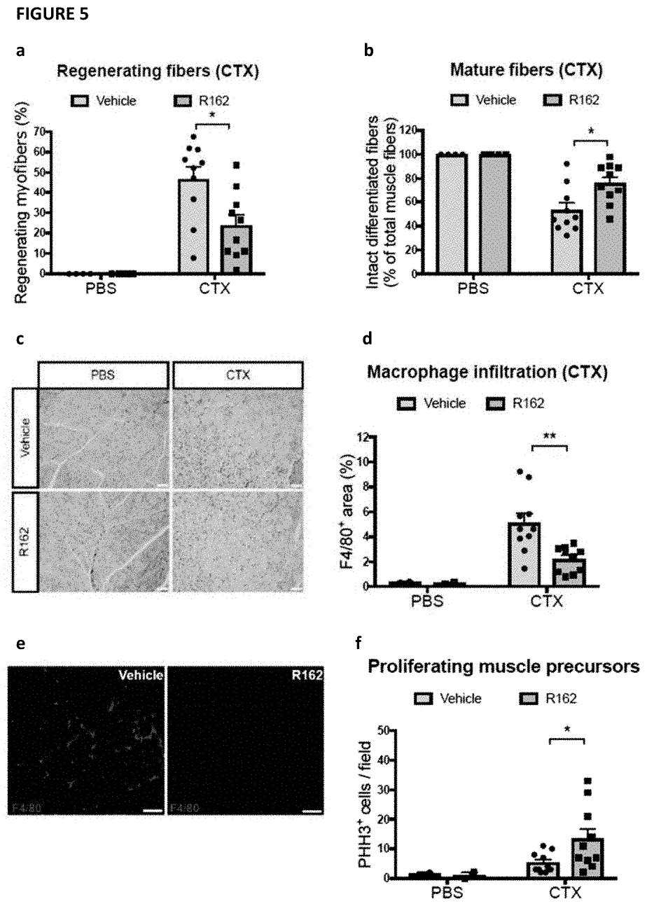

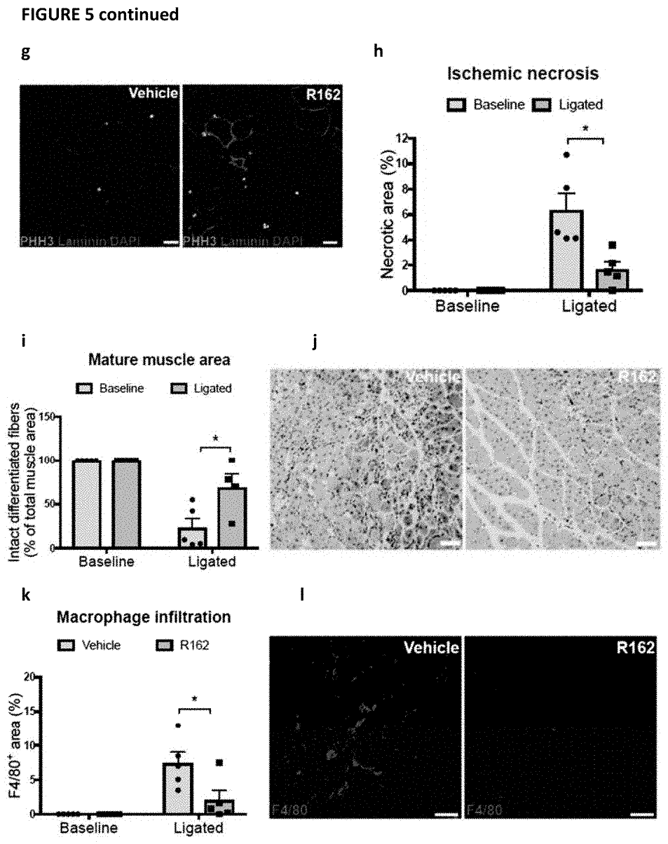

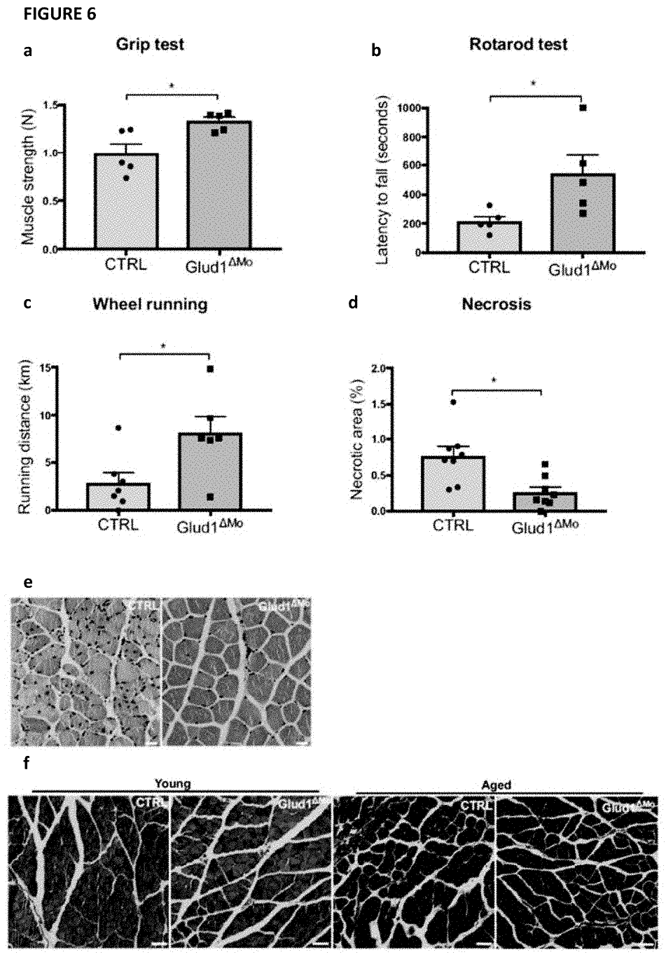

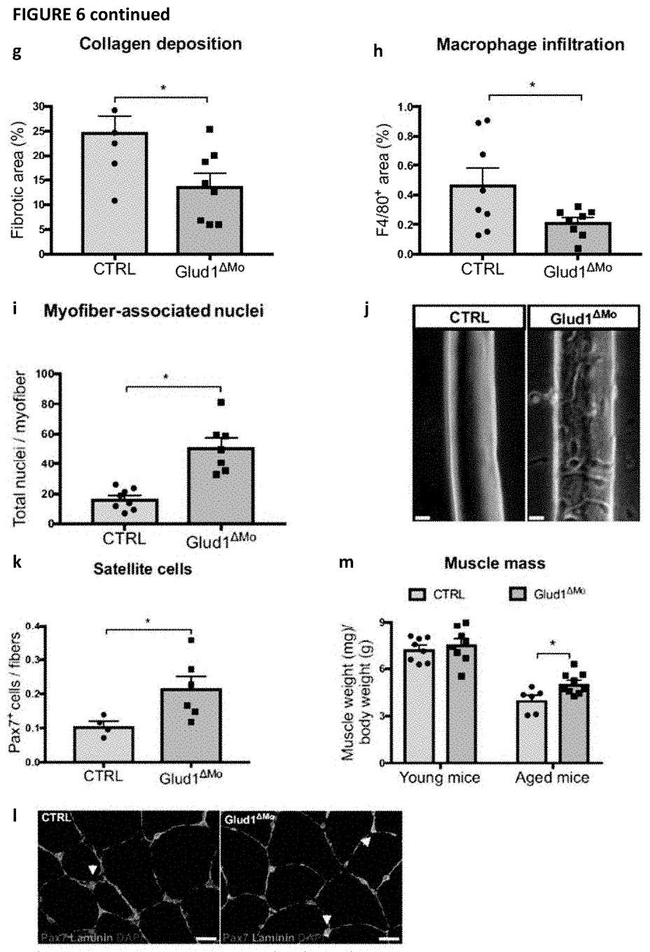

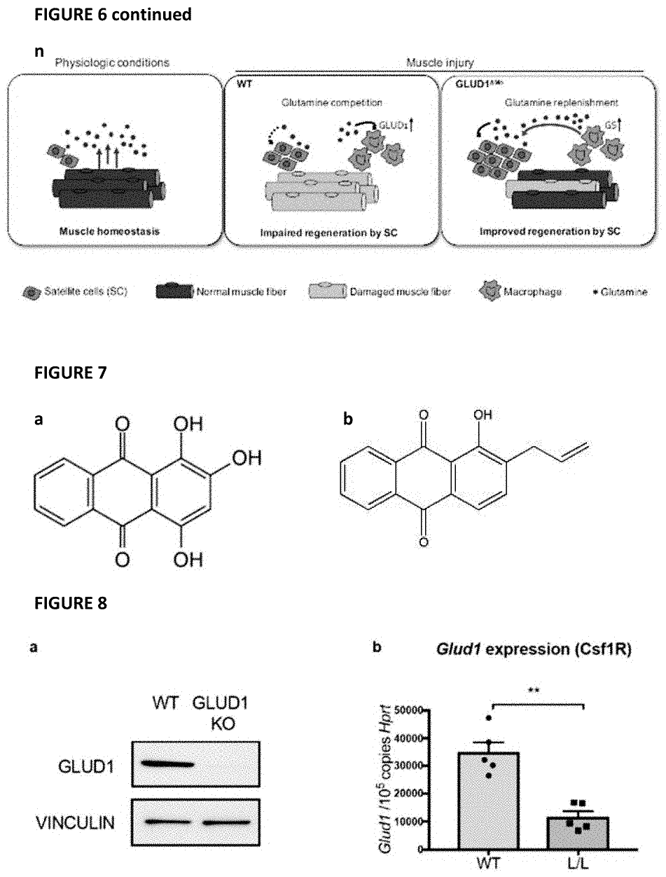

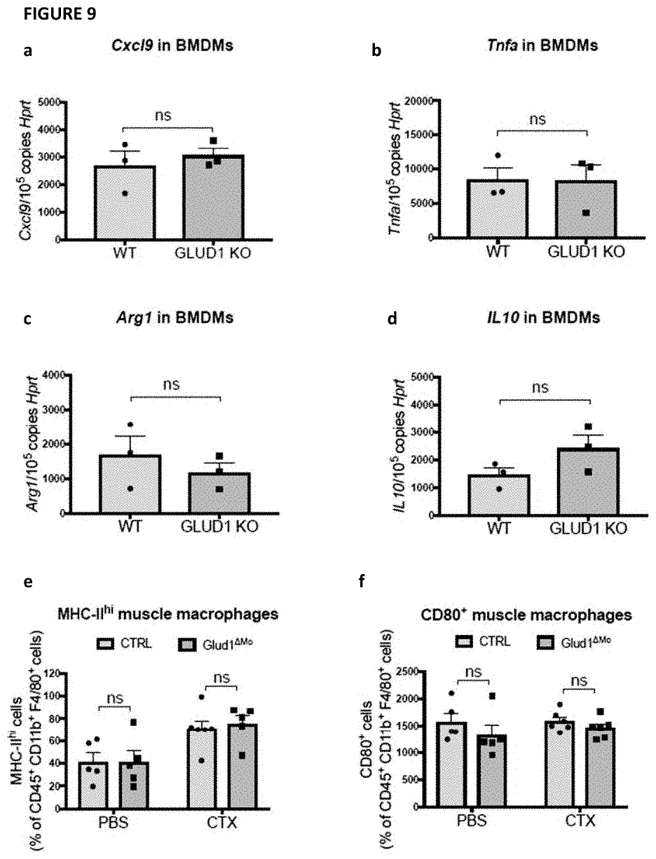

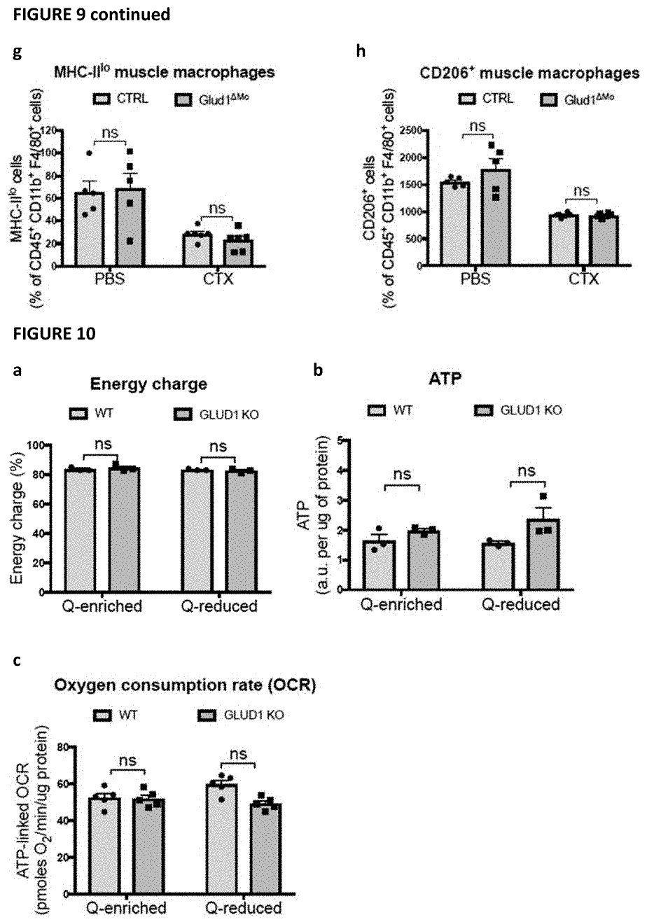

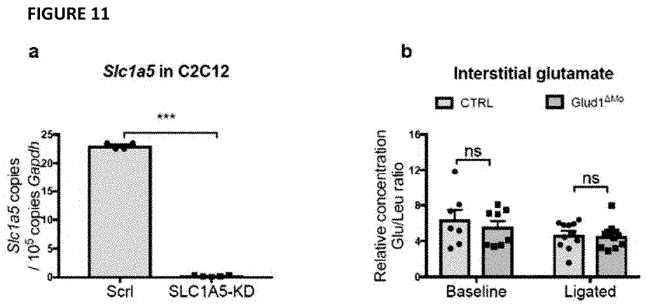

To understand the role of glutamine metabolism in macrophages, we intercrossed Glud1 floxed mice (Glud1^{L/L}) with the myeloid cell deleter LysM:Cre trangenic line, thus obtaining mice lacking GLUD1 in macrophages (FIG. 8a), from here on referred as Glud1^{ΔMo} mice. Compared to their littermate controls (CTRL), Glud1^{ΔMo} mice displayed a similar blood count (Table 1) and were overtly normal at baseline. Since skeletal muscle represents about 40% of the body weight and plays a pivotal role in glutamine metabolism/production (Shimomura et al. 2004, J Nutr 134:1583S-1587S), we considered to perturbate its homeostasis by provoking an acute inflammatory damage. To this end, we injected cardiotoxin (CTX) in the tibialis anterior (TA), a known snake venom toxin able to induce myolysis of myofibers, which eventually triggers the regeneration events (Guardiola et al. 2017, J Vis Exp 119:e54515). Six days after CTX injection, we noticed that muscle necrosis was lower in Glud1^{ΔMo} than in CTRL mice (FIG. 1 a,c). As a consequence of a presence of a damage, regenerative fibers (characterized by the presence of central nuclei) were present in TA muscles of CTRL mice but strongly reduced in those from Glud1^{ΔMo} mice (FIG. 1 b,c). Reduced muscle damage in Glud1^{ΔMo} mice after CTX injection translated into a faster functional recovery as assessed in a voluntary wheel running test (FIG. 1 d). Muscle histology and physical activity at baseline were comparable in both genotypes (FIG. 1 c,d). We then translated our findings into a more physiologically and clinically relevant mouse model of muscle ischemia, which causes extensive necrosis of the calf muscles because of poor blood supply (Niiyama et al. 2009, J Vis Exp 23:e1035). Remarkably, 14 days after femoral artery ligation ischemic necrosis of the calf was 95% lower in Glud1^{ΔMo} than CTRL mice (FIG. 1 e,g). Also in this case, this was associated with on-going regeneration in CTRL but no longer in Glud1^{ΔMo} mice (FIG. 1 f,g). TUNEL and dehydroethidium (DHE) stainings indicated respectively that myofibers in CTRL mice had signs of apoptosis and oxidative damage at end stage. Contrariwise, loss of GLUD1 in myeloid cells resulted in reduced muscle apoptosis and oxidative damage (FIG. 1 h-o). The use of a CSFR1:Cre-ERT deleter line allows the excision of floxed alleles specifically in macrophages following tamoxifen administration (Palmieri et al. 2017, Cell Rep 20:1654-1666; Cassaza et al. 2013, Cancer Cell 24:695-709). Inducible deletion of Glud1^{L/L} in Glud1^{L/L}×CSFR1:Cre-ERT mice (FIG. 8b) reduced CTX-induced muscle necrosis by 45% when compared to controls (Glud1^{WT/WT}×CSFR1:Cre-ERT) (FIG. 1 p,r), with associated regeneration features (FIG. 1 q,r). Overall our data indicate that GLUD1 loss in macrophages is strongly improving the outcome of acute muscle degeneration.

TABLE 1
The values show the hematological parameters (mean ± SEM)
in control (LysM:Cre × Glud1WT/WT; CTRL in short) and
myeloid cell specific Glud1 deficient mice (LysM:Cre ×
Glud1L/L; Glud1DMo in short). Data are pooled from 2 independent
cohorts of 5 mice per condition, total n = 10 per each condition.

| Parameter | CTRL | Glud1^{ΔMo} |
|---|---|---|
| WBC (K/μl) | 4.51 ± 1.09 | 4.56 ± 0.88 |
| NEU (%) | 11.4875 ± 2.11 | 12.3 ± 2.80 |
| LYM (%) | 84.95 ± 2.38 | 83.61 ± 2.45 |
| MON (%) | 1.82 ± 0.44 | 2.01 ± 0.70 |
| EOS (%) | 0.88 ± 0.32 | 0.93 ± 0.32 |
| BAS (%) | 0.16 ± 0.08 | 0.18 ± 0.08 |
| RBC (M/μl) | 8.84 ± 0.32 | 8.51 ± 0.47 |
| PLT (K/μl) | 630.4 ± 99.92 | 692 ± 81.10 |

Abbreviations: white blood cell (WBC), neutrophil (NEU), lymphocyte (LYM), monocyte (MON), eosinophil (EOS), basophil (BAS), red blood cell (RBC), platelet (PLT).

### 2. GLUD1 Deficiency in Macrophages Favours Muscle Repair by Activation of SC

We then questioned if the improved disease outcome observed in Glud1^{ΔMo} mice was due to reduced damage upon injury (muscle protection) or to accelerated muscle healing (muscle repair). To address this question, we performed a time course analysis and found that in both the CTX and ischemic models, the initial damage was comparable in both genotypes but was gradually and more promptly ameliorated in Glud1^{ΔMo} than in CTRL mice as indicated by morphometric quantification of necrosis in H&E sections of crural muscles and by the faster resolution of inflammation (FIG. 2 a-f). Importantly, in vitro migration of CTRL and GLUD1-deficient macrophages was comparable (FIG. 2g), further suggesting that the quicker resolution of F4/80⁺ infiltrates in the injured muscles of Glud1^{ΔMo} mice is a consequence of muscle healing. An important aspect related to muscle healing is the perfusion and vascularization of the muscle. By Doppler analysis, we saw that perfusion following femoral artery ligation was reduced by 80% in both CTRL and Glud1^{ΔMo} mice and it equally improved over the time (FIG. 2h). This was consistent with comparable vessel number prior and after muscle damage in both genotypes (FIG. 2i). We thus focused our attention on the number and activation of satellite cells (SC). In a time-course fashion in both CTX-injected and ischemic muscles, we showed that at an early time point (i.e., when necrosis and inflammation are comparable in both CTRL and Glud1^{ΔMo} mice) proliferating muscle precursors, adjacent to the basal lamina (Laminin⁺) and positive for phospho-histone H3 (PHH3), which is also expressed by activated SC (Hosoyama et al. 2011, J Biol Chem 286:19556-19564; Marti et al. 2013, J Cell Sci 126:5116-5131; Naito et al. 2016, PLoS Genet 12:e1006167), were twice more abundant in Glud1^{ΔMo} than in CTRL muscles (FIG. 2 j-m). This correlated with an increased expression in whole muscle lysates of Pax7, a specific marker for SC (Costamagna et al. 2015, Curr Gene Ther 15:348-363; FIG. 2 n-q). Moreover, analysis of total nuclei associated to freshly isolated single fibers, a readout of muscle precursors (Fry et al. 2015, Nat Med 21:76-80), were enriched in Glud1^{ΔMo} versus CTRL injured mice (FIG. 2 r,s). While basal levels of SC were comparable (FIG. 2 n-q), our analyses showed that the myogenic potential of muscles from Glud1^{ΔMo} mice is higher than those from CTRL mice.

### 3. GLUD1 Deficiency in Macrophages Favours Glutamine Synthesis and Release

Starting from the observations that, compared to WT cells, both in vitro and FACS-sorted GLUD1 deficient macrophages (both at baseline and upon muscle damage) did not display overt differences in the expression of some key phenotypic markers (FIG. 9 a-h) that have been previously associated with improved healing functions (Novak & Koh 2013, Am J Pathol 183:1352-1363; Rigamonti et al. 2014, Biomed Res Int 2014: Article ID 560629), we focused on their metabolic pattern since it is known that metabolism of a certain cell type can affect the behaviour and biology of neighbouring cells. Firstly, we measured glutamine oxidation in glutamine (Q)-enriched or glutamine (Q)-reduced medium by measuring the amount of ¹⁴CO₂ released from supplemented radiolabelled glutamine. In CTRL macrophages, glutamine oxidation (QO) was 73% lower in glutamine-reduced medium than in glutamine-enriched conditions. Glutamine oxidation in GLUD1-deficient macrophages was lower in both culture conditions when compared to CTRL cells (FIG. 3a). Nevertheless, total energy charge, ATP production, and ATP-linked oxygen consumption rate (OCR) were all comparable in both genotypes either in glutamine-enriched or in glutamine-reduced medium (FIG. 10 a-c). Despite decreased glutamine oxidation in GLUD1-deficient macrophages, total 2-oxoglutarate was also comparable in both genotypes (FIG. 3b), overall arguing that other carbon sources are compensating for impaired glutamine anaplerosis in absence of GLUD1.

However, the drop in glutamine oxidation observed in GLUD1-deficient macrophages was associated with a marked increase in intracellular and extracellular glutamine, in both glutamine-enriched and, to a higher extent, glutamine-reduced media (FIG. 3 c,d). Altogether, these observations led us to speculate that GLUD1-deficient macrophages did not consume less glutamine but were rather able to produce more of this amino acid. It is well established that glutamine synthetase (GS) has the important function to prevent ammonia accumulation in cells by catalyzing the condensation of glutamate and ammonia in glutamine (Hakvoort et al. 2017, Hepatology 65:281-293). Firstly, we proved that glutamine uptake and glutamine incorporation into glutamate were comparable in CTRL and GLUD1-deficient macrophages (FIG. 3 e,f). Secondly, we showed that, in glutamine-reduced media, ¹⁵NH₄⁺ was more promptly used by GLUD1-deficient macrophages than the CTRL cells, resulting in increased intracellular and extracellular ¹⁵N-labelled glutamine (FIG. 3g). This increase in GS activity was accompanied by higher GS protein levels in GLUD1-deficient macrophages, already in presence of glutamine but even at higher extent under glutamine starvation (FIG. 3 h,i). As expected, GS was induced in CTRL macrophages upon glutamine starvation but this induction was stronger in GLUD1-deficient macrophages (FIG. 3 h,i). In line with the fact that GLUD1 is controlling the reverse reaction that gives glutamate from 2-oxoglutarate (and being glutamate the substrate of GS), we noticed that glutamine deprivation also induced the protein levels of GLUD1 in CTRL macrophages (that was absent in the knockout) (FIG. 3 j,k).

To derive glutamate under glutamine starvation, glucose becomes an important carbon source in CTRL macrophages (FIG. 3l) and glucose incorporation into glutamate was enhanced in GLUD1-deficient macrophages (FIG. 3m). Since GLUD1 is catalysing a reversible reaction, the formation of glutamate from glucose implies the conversion of 2-oxoglutarate into glutamate by GLUD1. In absence of GLUD1, this reaction was likely compensated by the increased activity of aspartate aminotransferase (AST), which produces glutamate from 2-oxoglutarate and aspartate as a nitrogen-group donor in glutamine-reduced medium (FIG. 3n). Consistent with the enhanced conversion of glutamate into glutamine by GS in GLUD1-deficient macrophages, extracellular glutamine production in GLUD1-deficient macrophages was rescued back to the CTRL levels when GS was inhibited by methionine sulfoximine (MSO) (FIG. 3o). Overall, our data show that GLUD1 deficiency in macrophages did not affect the energy status, but rather rerouted glutamate fate towards glutamine production and release.

### 4. Glutamine Release by GLUD1-Deficient Macrophages Reinforces Myogenic Differentiation

To link the metabolic features observed in GLUD1-deficient macrophages to the myogenic potential of muscle precursors upon muscle damage, we cultured the myoblast cell line C2C12 in differentiation medium (i.e. 2% horse serum) (Blau et al. 1983, Cell 32:1171-1180), to induce myoblast fusion and the formation of multinucleated fibers in the presence of glutamine or in a glutamine-deprived medium (as achieved by serum dialysis). Myotube formation was the highest in glutamine rich medium and strongly hindered by the absence of glutamine. When adding CTRL macrophages to C2C12 cells cultured in glutamine-enriched conditions, the myogenic potential was reduced to a similar extent as in absence of glutamine. The presence of Glud1^{ΔMo} macrophages co-cultured with C2C12 myoblasts resulted in larger myotubes but only when glutamine was limiting (FIG. 4 a,b). Similar results were obtained especially under glutamine deprivation when we exposed C2C12 cells to media conditioned for 48 hours by GLUD1-deficient macrophages (FIG. 4 c,d), supporting the idea that a soluble molecule, possibly a limiting metabolite, was released into the extracellular milieu by macrophages and was responsible for myogenic differentiation. To investigate whether this was due to glutamine release, we brought together in culture GLUD1-knockout macrophages and SLC1A5-deficient C2C12 cells (obtained by using a gRNA against Slc1a5), which have reduced glutamine uptake because of their reduced expression of the glutamine transporter SLC1A5 (FIG. 4e, FIG. 11a). Impairment of glutamine uptake in myoblasts abrogated the advantage offered by GLUD1-knockout macrophages on myoblast differentiation (FIG. 4 f,g). These observations support the idea that GS induction in macrophages lacking GLUD1 and consequent glutamine release may be causative of in vivo SC activation during injury. First, we measured the amount of glutamine in the extracellular fluid of normal and injured muscles from CTRL and Glud1^{ΔMo} mice. Despite basal levels of glutamine were equally high in CTRL and Glud1^{ΔMo} mice, in a restrictive situation, myeloid deficiency of GLUD1 increased glutamine availability in the extracellular milieu (FIG. 4 h,i). No differences were observed when measuring the extracellular abundance of glutamate both in vitro (in the culture media of CTRL or Glud1-deficient macrophages) (FIG. 4j) and in vivo (in the interstitial fluid from CTRL or Glud1^{ΔMo} muscles) (FIG. 4k, FIG. 11b). Secondly, we assessed muscle damage in CTRL and Glud1^{ΔMo} mice that were competent or knockout for GS in their myeloid lineage. Double knockout of GLUD1 and GS completely abrogated the reduction in necrosis, observed in Glud1^{ΔMo} mice (FIG. 4l). Finally, we induced muscle injury in CTRL and Glud1^{ΔMo} mice by CTX administration and simultaneously inhibited the glutamine transporter SLC1A5 by oral administration of gamma-L-Glutamyl-p-Nitroanilide (GPNA) (Hassanein et al. 2015, Int J Cancer 137:1587-1597). We prove that the inhibitor blocked glutamine uptake in C2C12 cell by 75% (FIG. 4e). In vivo, GPNA abolished the abundance of proliferating myogenic precursors in Glud1^{ΔMo} muscles (FIG. 4m). Altogether, our data show that interstitial glutamine is required for SC activation. Infiltrating macrophages compete for glutamine and impair SC activation. GLUD1 deficiency prompts GS activity and breaks this competition. In turn, GLUD1-deficient macrophages will instead release their excessive production of glutamine therefore mitigating glutamine starvation as it occurs in acute muscle damages, and altogether allowing a better muscle regeneration via SC activation and proliferation.

### 5. Systemic Inhibition of GLUD1 by the R162 Compound Improves Muscle Regeneration

To assess the pharmacologic value of our findings, we evaluated whether the previously characterized GLUD1 inhibitor R162 (FIG. 7, Jin et al. 2015, Cancer Cell 27:257-270), shown to have a therapeutic effect on tumor growth, could offer in a setting of muscle degeneration a similar advantage as macrophage-specific GLUD1 deficiency. Following CTX administration, we proved that bi-daily oral gavage of R162 strongly improved the quality of the muscle (FIG. 5 a-c) and reduced inflammation (FIG. 5 d-e). In line, R162 increased by 60% the number of PHH3⁺ cells upon damage (FIG. 5 f-g). Similar results were obtained when assessing the therapeutic effect of R162 on muscle ischemia with improved repair of the necrotic damage (FIG. 5 h-j) and resolution of the inflammatory response (FIG. 5 k,l). Systemic administration of R162 will block GLUD1 in all the cells. These data suggest that GLUD1 is dispensable in SC (and muscle precursors in general) as also supported by a previous report (Ryall et al. 2015, Cell Stem Cell 16:171-183). However, by blocking GLUD1 in macrophages, R162 is likely to break glutamine competition between SC and macrophages, resulting in an advantageous effect during muscle regeneration.

### 6. Macrophage GLUD1 Deficiency Ameliorates Age-Related Muscle Wasting

Finally, we wondered if chronic muscle degeneration could take advantage from GLUD1 deficiency in macrophages. To this end, we decided to analyse how aging can affect muscle histology and function in CTRL and Glud1^{ΔMo} mice. At 16-18 months of age, Glud1^{ΔMo} mice displayed improved muscle functionality as assessed in a grip test (mostly assessing muscle strength) (FIG. 6a), in a rotarod test (mostly assessing motor coordination) (FIG. 6b), and in a voluntary wheel running test (mostly assessing physical performance and endurance) (FIG. 6c). From a histological point of view, necrosis (FIG. 6 d,e), and fibrotic deposition (FIG. 6 f,g), were reduced in muscles from Glud1^{ΔMo} than those from CTRL mice, which was accompanied by a milder inflammatory infiltrate (FIG. 6h). When counting the total nuclei associated to single fibers isolated from Glud1^{ΔMo} muscles, these were 3 times more abundant than in CTRL specimens (FIG. 6 i,j). Double number of Pax7⁺ cells in Glud1^{ΔMo} versus CTRL aged mice (FIG. 6 k,l), further corroborated the observation that, within the stem cell compartment, SC were more abundant in the absence of GLUD1 in macrophages. These muscle adaptations were in line with a higher muscle weight index in Glud1^{ΔMo} versus CTRL mice (FIG. 6m). Overall, these data suggest that GLUD1 impairment in macrophages mitigates the outcome of muscle aging preserving both loss of muscle mass and function.

### 7. Adoptive Transfer of Macrophages Engineered Toward GLUD1 Inhibition

Bone marrow derived macrophages are maintained in culture, optionally in the presence of a pharmacologic GLUD1-inhibitor. Macrophages are then genetically engineered or re-directed such as to knock out the GLUD1 gene, or to introduce a vector or other genetic construct comprising an inducible promotor operably linked to a cassette allowing expression of a genetic or nucleotide based GLUD1-inhibitor (e.g. miRNA, shRNA, antisense RNA, ribozyme). In the latter case the macrophages are conditionally expressing a GLUD1 inhibitor. The engineered macrophages are subsequently transferred into the subject, such as in the subject's muscle(s) or intravenously, such as to treat muscle wasting, muscle wasting disease or muscle atrophy as described hereinabove. In case of macrophages engineered towards inducible GLUD1-inhibition, the expression inducing compound is administered at an appropriate timepoint to the subject having received the engineered macrophages. The transfer (adoptive cell transfer) can be autologous or heterologous. Adoptive macrophage transfer has been described in the literature (e.g. Ma et al. 2015, Brain Behaviour Immunity 45:157-170; Parsa et al. 2012, Diabetes 61:2881-2892; Wang et al. 2007, Kidney Int 72:290-299; Zhang et al. 2014, Glia 62:804-817).

### Materials and Methods

Animals:

Floxed GLUD1 MGI:3835667)(Carobbio et al. 2009, J Biol Chem 284:921-929) and GS floxed (Glul^{tm3Whla}, MGI:4462791)(He et al. 2010, Glia 58:741-754) mice, both in a C57BL/6 background, were obtained respectively from Pierre Maechler (University of Geneva, Switzerland) and Wouter H. Lamers (Academic Medical Center, Amsterdam, Netherlands). We generated Glud1^{L/L}×CSFR1:Cre-ERT mice by intercrossing Glud1 floxed mice with the tamoxifen-inducible, macrophage-specific CSFR1:Cre-ERT deleter mouse line (a gift of Jeffrey W. Pollard, University of Edinburgh, UK). Glud1^{WT/WT}×CSFR1:Cre-ERT littermates were used as controls. Acute deletion of Glud1 in macrophages was obtained by daily i.p. injection of tamoxifen (0.05 mg per gram of body weight) for 5 days before and during cardiotoxin (CTX) (Latoxan) induced injury. Control mice were treated with tamoxifen according to the same protocol. All mice used for ischemia and CTX experiments were on a C57BL/6 background between 8 and 15 weeks old, while 16-18 months old mice were used for the aged-related experimental setting. Mice were used without specific gender selection. In all experiments, littermate controls were used. Housing and all experimental animal procedures were approved by the Institutional Animal Care and Research Advisory Committee of the KU Leuven.

Cardiotoxin Muscle Injury:

Mice were anaesthetized with isoflurane and 50 μl of 10 μM CTX was injected in the tibialis anterior (TA) muscle. Muscles were harvested for analysis at different time points post-injury (day 1 and day 6). In vivo GLUD1 inhibition was achieved by bi-daily gavage of R162 (Focus Biomolecules) at 0.6 mg/mouse. Mice were pretreated 1 day before CTX injection or ischemic ligation. Afterwards, mice continued to receive daily treatment until their sacrifice. To inhibit SLC1A5, mice were treated 3 times per day with 500 mM GPNA (Sigma-Aldrich). CTX was injected 1 hour after the first gavage, and mice were sacrificed 24 hours afterwards.

Hindlimb Ischemia:

To induce acute hindlimb ischemia and greatly prevent flow redirection into the collateral circulation, which leads to severe muscle necrosis, unilateral or bilateral ligations of the high femoral artery were performed without damaging the nervus femoralis as previously described (Padgett et al. 2016, J Vis Exp 112: e54166). Control mice were subjected to a sham operation that did not involve the ligation of the femoral artery. Functional perfusion measurements were performed using a Lisca PIM II camera (Gambro) (Takeda et al. 2011, Nature 479:122-126).

Voluntary Wheel Running Test:

Muscle functionality was assessed in a voluntary wheel running test, mice were housed separately in cages equipped with voluntary running wheels. Duration and velocity of voluntary wheel running was recorded by a computer system, after 1 week training.

Rotarod Test:

Whole body mobility and coordination was assessed by rotarod performance. Following a 5-min acclimatization in the rest room, mice were placed on the rod (Biological research apparatus), which was rotating at an initial speed of 4 rpm. The speed was increased gradually from 4 rpm to 40 rpm within 5 mins and latency to fall on to a soft pad was recorded. The test was repeated twice more, with 15 minutes between tests. After 3 days training, latency to fall was calculated over the 3 trials.

Grip Test:

Muscle strength was assessed in a grip test. The strength was measured by pulling backwards the mice with a continuous movement, when the mice is holding firmly to the grip. The test was repeated twice more, with 15 minutes between tests. Results was calculated over the 3 trials.

Bone Marrow-Derived Macrophages (BMDMs):

Macrophages were derived from bone marrow precursors as described before (Casazza et al. 2013, Cancer Cell 24:695-709). Briefly, bone marrow cells (1.6×10⁶ cells/ml) were cultured in a volume of 6 ml in a 10 cm Petri dish in DMEM supplemented with 20% FBS and 30% L929 conditioned medium as a source of M-CSF. After 3 days of culture, an additional 3 ml of differentiation medium was added. At day 7, macrophages were harvested with ice cold Ca2⁺ and Mg2⁺-free PBS. The cells obtained were uniformly macrophages as assessed by FACS, using the pan-macrophage marker F4/80. When indicated, GS inhibition in cultured BMDMs was achieved by adding 1 mM L-methionine-SR-sulfoximine (MSO; Sigma) to the medium for 48 hours.

BMDM Migration Assay:

Migration of BMDMs was assessed by using a 8-μm-pore Transwell permeable plate (Corning Life Science). Lower chambers were pretreated with DMEM at 20% FBS for 30 min, BMDMs were harvested and then seeded in the upper chamber (2.5×10⁵ cells in 200 μl of DMEM at 2% FBS). After 4 h incubation, migrated cells were fixed with 4% paraformaldehyde, stained with 5 mg/mL crystal violet/20% methanol and counted under the microscope.

Glutamine Uptake:

BMDMs were incubated in M199 medium (Gibco) supplemented with 10% FBS and 0.5 μCi/ml [U-¹⁴C]-glutamine for 30 min at 37° C. Cells were lysed in 1N NaOH and the radioactivity was measured by liquid scintillation counting.

Glutamine Oxidation:

BMDMs were incubated for 6 h in M199 with 10% FBS containing 0.5 μCi/ml [U-¹⁴C]-glutamine. Thereafter, 250 μl of 2 M perchloric acid was added to each well to stop cellular metabolism and wells were immediately covered with a 1× hyamine hydroxide-saturated Whatman paper. Overnight absorption of ¹⁴CO₂ released during the oxidation of glutamine into the paper was performed at RT and radioactivity in the paper was determined by liquid scintillation counting.

A(X)P Detection by LC-MS:

2×10⁶ BMDMs were lysed in 300 μl extraction buffer (50:30:20 mix of methanol:acetonitrile:10 mM Tris pH 9.3). Following extraction, samples were centrifuged for 10 min at 20×10³×g (at 4° C.). The supernatant was transferred to a vial. 35 μl was loaded onto an Ultimate 3000 UPLC (Thermo Scientific, Bremen, Germany) equipped with a ZIC-pHILIC column (2.1×150 mm, 5 μm particle size, cat #1.50460.0001, Merck, Darmstadt, Germany) in line connected to a Q Exactive mass spectrometer (Thermo Fisher Scientific). A linear gradient was carried out starting with 90% solvent A and 10% solvent B. From 2 to 20 minutes the gradient changed to 80% B and was kept at 80% until 23 min. Next a decrease to 40% B was carried out to 25 min, further decreasing to 10% B at 27 min. Finally 10% B was maintained until 35 min. The solvent was used at a flow rate of 200 μl/min, the columns temperature was kept constant at 25° C. The mass spectrometer operated in negative ion mode, settings of the HESI probe were as follows: sheath gas flow rate at 35, auxiliary gas flow rate at 10 (at a temperature of 260° C.). Spray voltage was set at 4.8 kV, temperature of the capillary at 300° C. and S-lens RF level at 50. A full scan (resolution of 140.000 and scan range of m/z 70-1050) was applied. For the data analysis we used an in-house library and metabolites of interest were quantified (area under the curve) using the XCalibur 4.0 (Thermo Scientific) software platform. The energy charge was calculated as ([ATP]+1/2[ADP])/([ATP]+[ADP]+[AMP]).

Oxygen Consumption:

1.5×10⁴ BMDMs were incubated overnight on Seahorse XF24 tissue culture plates (Agilent). During the assay, the medium was replaced by unbuffered DMEM supplemented with 5 mM D-glucose and 2 mM L-glutamine, pH 7.4. The measurement of oxygen consumption was performed at 6 min intervals (2 min mixing, 2 min recovery, 2 min measuring) using the Seahorse XF24 analyzer. Inhibitors were serially injected at the following concentrations: oligomycin (1 μM), FCCP (fluoro-carbonyl cyanide phenylhydrazone, 1.5 μM), antimycin A (1 μM) (all from Sigma-Aldrich).

¹³C and ¹⁵N Tracing Experiments:

For ¹³C and ¹⁵N tracing experiments, cells were incubated with [U-¹³C]-L-glutamine (2 mM), [U-¹³C]-D-glucose (5 mM), ¹⁵NH₄Cl (2 mM) or [¹⁵N, ¹³C4]-aspartate (1 mM) for 48 hours (confirmation of steady state), respectively (Cambridge Isotope Laboratories).

Sample Preparation for Mass Spectrometry:

BMDMs were scraped in 80% methanol and phase separation was achieved by centrifugation at 4° C. Methanol-water phase containing polar metabolites was separated and dried using a vacuum concentrator. The dried metabolite samples were stored at −80° C. Isotopomer distributions and metabolite levels were measured with a 7890A GC system (Agilent Technologies) combined with a 5975C Inert MS system (Agilent Technologies).

Interstitial Fluid:

Intact muscle tissue (TA muscle for the CTX model and crural muscle for the ischemia model) was placed into test tubes with perforated bottom. 204 of 0.9% NaCl solution pH 7.4 was added to the tissue sample. Interstitial fluid was collected by centrifugation (110 g, 10 min, 4° C.). Protein within the interstitial fluid was precipitated using −20° C. cold methanol/water-mix (5:3) and centrifuged (21130 g, 2 min, 4° C.). The supernatant was dried using a vacuum centrifuge and derivatized for mass spectrometry analysis.

Metabolites Quantification by LC-MS/MS:

For mass spectrometry analysis of glutamate and glutamine, 2×10⁶ cell pellets were washed twice in PBS and extracted in 500 μl of 80% methanol (80-20, methanol-water). Upon extraction, samples were centrifuged at 20.000×g for 15 min and the supernatant was dried using a vacuum centrifuge. Twenty-five μl of a 2% methoxyamine hydrochloride solution (20 mg dissolved in 1 ml pyridine) were added to the dried pellet and the tubes were then placed at 37° C. for 90 min. Then 75 μl of N-tert-Butyldimethylsilyl-N-methyltrifluoroacetamide with 1% N-tert-Butyldimethyl-chlorosilane (Sigma-Aldrich, Bornem, Belgium) was added and the reaction was carried out for 30 min at 60° C. Reaction mixtures were then centrifuged for 15 min at 20,000×g at 4° C. in order to remove insolubilities, the supernatant was transferred to a glass vial with conical insert (Agilent). GC-MS analyses were performed using an Agilent 7890A GC equipped with a HP-5 ms 5% Phenyl Methyl Silox (30 m-0.25 mm i.d.-0.25 μm; Agilent Technologies, Santa Clara, Calif., USA) capillary column, interfaced with a triple quadruple tandem mass spectrometer (Agilent 7000B, Agilent Technologies) operating under ionization by electron impact at 70 eV. The injection port, interface and ion source temperatures were kept at 230° C. Temperature of the quadrupoles was maintained at 150° C. The injection volume was 1 μl, and samples were injected at 1:25 split ratio. Helium flow was kept constant at 1 ml/min. The GC oven temperature was held at 60° C. for 3 min, increased to 300° C. at 9° C./min, and kept for 2 min. The mass spectrometer operated in SIM mode, glutamine and glutamate were determined from the m/z 341.2 and 342.2 respectively. For the quantification we used the Agilent Masshunter Quan tool.

FACS Analysis of Muscle Macrophages:

TA muscles were dissected, dissociated mechanically, and digested using 800 U/mL collagenase II (10 ml per sample) for 1 hour at 37° C., centrifuged and resuspended with 1000 U/mL collagenase II (1 ml per sample) and 11 U/ml Dippase (1 ml per sample) solution followed by incubation for 30 min at 37° C. The digested tissue was filtered using a 40 μm pore sized mesh and cells were centrifuged 5 min at 500 g. For flow sorting, the myeloid cell population in the single cell suspension, and when appropriate in flushed bone marrow cells, was enriched by coating with CD11b-conjugated magnetic beads (MACS, Miltenyi Biotec) and separation through magnetic columns (MACS, Miltenyi Biotec). Cells were resuspended in FACS buffer (PBS containing 2% FBS and 2 mM EDTA) and incubated for 15 minutes with Mouse BD Fc Block™ purified anti-mouse CD16/CD32 mAb (BD-pharmingen) and stained with the following antibodies for 30 minutes at 4° C.: viability dye, anti-CD45 (30-F11), anti-CD11b (M1/70), anti-F4/80 (BM8), anti-MHCII (M5/114.15-12), anti-CD11c (NK418), anti-CD80 (B7-1) and anti-206 (MR5D3), all from eBioscience. Cells were subsequently, washed and resuspended in cold FACS buffer before FACS analysis or flow sorting by a FACS Verse or FACS Aria (BD Biosciences), respectively. FMO controls were performed in all the staining and used for the proper gating of the positive populations in all the analysis.

Histology and Immunostainings:

7 μm-thick cryosections were obtained by using a Leica cryostat from frozen muscles collected in optimal cutting temperature compound (OCT). Alternatively, tibialis anterior and calf muscles (gastrocnemius) were fixed in 2% paraformaldehyde, dehydrated, embedded in paraffin, and sectioned at 7 μm thickness. Necrotic and regenerating muscle fibers, were detected by H&E staining as previously described (Takeda et al. 2011, Nature 479:122-126) and analysed by ImageJ software. After deparaffinization and rehydration, muscle sections were permeabilized by a solution containing 1% BSA, 0.2% Triton-X in PBS 30 min at RT and blocked with Donkey serum (Sigma)1 h at RT. An antigen retrieval step was performed for Pax7 immunofluorescence after the permeabilization, placing the slides in pH 6.1 citrate buffer (Dako) at 92° C. for 20 min. Slides were incubated overnight at 4° C. with primary antibodies: rat anti-F4/80 (Serotec), rabbit anti-laminin (Sigma-Aldrich), rabbit anti-phospho-histone H3 (pSer10) (Millipore), mouse anti-Myosin (DSHB), mouse anti-Pax7 (DSHB), rat anti-CD34 (BD Pharmingen). Appropriate secondary antibodies were used: Alexa 488 or 568 conjugated secondary antibodies (Molecular Probes) 1:200, biotin-labeled antibodies (Jackson Immunoresearch) 1:300 and, when necessary, TCA fluoricine, TSA Plus Cyanine 3 or Cyanine 5 System amplification (Perkin Elmer, Life Sciences) were performed according to the manufacturer's instructions. Whenever sections were stained in fluorescence, ProLong Gold mounting medium with or without DAPI (Invitrogen) was used. Microscopic analysis was performed by Olympus BX41 microscope and CellSense imaging software.

In Vitro BMDMs-C2C12 Co-Cultures:

WT and SLC1A5-silenced C2C12 murine myoblast cell line were used for in vitro studies. Cells were cultured in Growth Medium (GM), containing DMEM (Gibco) supplemented with 10% Fetal Bovine Serum (FBS, Gibco), 2 mM glutamine, 100 units/ml penicillin and 100 μg/ml streptomycin. C2C12 myoblasts were cultured for no more than six passages in a humidified incubator in 5% CO₂ at 37° C.

6×10⁵ C2C12 cells were seeded in a 6-well plate and coltured in GM until they reached 70% of confluence, then GM was replaced with differentiation medium (DM, with glutamine or glutamine-free), supplemented with 2% horse serum (as such or dyalized for glutamine) for 2 days. At day 2 of myoblasts differentiation, 1.5×10⁵ BMDMs (WT or GLUD1 KO) were added to the differentiating myotubes. After 4 days of C2C12/BMDMs cocultures in DM conditions, cells were fixed in 4% PFA for 10 min at RT, then permeabilized in PBS with 1% BSA, 0.2% TritonX for 30 min and finally blocked in Donkey serum 1 h at RT. Afterwards, samples were incubated overnight at 4° C. with mouse anti-MF20 (DSHB) and subsequently incubated with AlexaFluor 568-conjugated donkey anti-mouse, washed with PBS and mounted on glass slides with Prolong Gold antifade mounting medium with DAPI (Thermo Fisher). Microscopic analysis was done by Olympus BX41 microscope and CellSense imaging software. Morpometric analyses were performed by ImageJ software.

Single Fibers Isolation:

Single myofibers and their satellite cells were isolated from TA muscles as previously described (Pasut et al. 2013, J Vis Exp 73:e50074). Briefly, intact muscles were dissected from tendon to tendon and digested with a solution containing 0.6% collagenase type I (Sigma) in DMEM (Dulbecco's modified Eagle's medium; high glucose, L-glutamine with 110 mg/ml sodium pyruvate) at 37° C. for 3 h. Afterward, individual fibers were gently separated from each others by pipetting and sequential washing in DMEM. Total nuclei were quantified in bright field microscopy.

Protein Extraction and Immunoblot:

Whole cell protein extraction was performed using extraction Buffer (20 mM Tris HCl, 150 mM NaCl, 1% Triton X-100, 10% glycerol, 5 mM EDTA) supplemented with Complete Mini protease inhibitor (Roche) and PhosSTOP Phosphatase Inhibitor (Roche). Proteins (50 μg) were separated by NuPAGE® Precast Gel (Thermofisher) and transferred electrophoretically to nitrocellulose membrane by iBlot System (Thermofisher). Nonspecific binding was blocked in Tris-HCl Buffered Saline Solution with 0.05% Tween-20 (TBST) containing 10% nonfat dry milk. The following antibodies were used: GLUD1 (Abcam), GS (Sigma-Aldrich), PAX7 (DSHB), vinculin (Sigma-Aldrich), and appropriate HRP-conjugated secondary antibodies (Santa Cruz). Signal was visualized by Enhanced Chemiluminescent Reagents (ECL, Invitrogen) or West Femto by Thermo Scientific according to the manufacturer's instructions and acquired by a LAS 4000 CCD camera with ImageQuant software (GE Healthcare).

Generation of SLC1A5-Deficient C2C12 Cells:

To generate stable C2C12 cells, deficient for SLC1A5, lentiCRISPR v2 vectors expressing the Cas9 along with a gRNA targeting the Slc1a5 locus (AATCCCTATCGATTCCTGTG, SEQ ID NO:3) or a scrambled gRNA (GAACAGTCGCGTTTGCGACT, SEQ ID NO:4) as control, were used. Transduced cells were selected with puromycin (4 μg/ml). The lentiCRISPR v2 was a gift from Feng Zhang (Addgene plasmid #52961). The gRNAs were cloned as described previously (Sanjana et al. 2014, Nat Methods 11:783-784).

qRT-PCR:

Cells were washed in PBS, collected in RLT buffer (Qiagen) and kept at −80° C. RNA was extracted with the RNeasy Micro kit (Qiagen) according to manufacturer's instructions. Reverse transcription to cDNA was performed with the SuperScript® III First Strand cDNA Synthesis Kit (Life Technologies) according to manufacturer's protocol. Pre-made assays were purchased from IDT. cDNA, primer/probe mix and TaqMan Fast Universal PCR Master Mix were prepared in a volume of 10 μl according to manufacturer's instructions (Applied Biosystems). Samples were loaded into an optical 96-well Fast Thermal Cycling plate (Applied Biosystems) and qRT-PCR were performed using an ABI Prism 7500 Fast Real-Time PCR System (Applied Biosystems). To detect genome editing (indel) of the Slc1a5 target locus at transcriptional level, we designed a specific primer set. The forward primer 5′-AATCCCTATCGATTCCTGTGG-3′ (SEQ ID NO:5) anneals to the gRNA cutting site whereas, the reverse primer 5′-GAACCGGCTGATGTGTTTGG-3′ (SEQ ID NO:6) anneals to a non-targeted coding region. Thus, mutations of the gRNA target site will disrupt the amplification of the target region. cDNA, primers and PowerUp SYBR Green Master Mix were prepared in a volume of 20 μl according to manufacturer's instructions (Applied Biosystems). Samples were loaded into an optical 96-well Fast Thermal Cycling plate (Applied Biosystems) and qRT-PCR was performed using an ABI Prism 7500 Fast Real-Time PCR System (Applied Biosystems).

Statistics:

Data entry and all analyses were performed in a blinded fashion. All statistical analyses were performed using GraphPad Prism software on mean values calculated from the averages of technical replicates. Statistical significance was calculated by two-tailed unpaired t-test on two experimental conditions or two-way ANOVA when repeated measures were compared, with p<0.05 considered statistically significant. Detection of mathematical outliers was performed using the Grubbs' test in GraphPad. Sample sizes for all experiments were chosen based on previous experiences. Independent experiments were pooled and analyzed together whenever possible as detailed in figure legends. All graphs show mean values±standard error of the mean (SEM).

1. A method for treating muscle wasting, muscle wasting disease, muscle atrophy, muscle injury, or muscle insult in a subject, the method comprising:
administering to the subject an inhibitor of GLUD1.

2. The method according to claim 1, wherein the administration of the inhibitor of GLUD1 inhibits, ameliorates, or halts the muscle wasting, muscle wasting disease or muscle atrophy; and
wherein the subject is an elderly subject, an immobile subject, or is at risk of developing muscle wasting, muscle wasting disease, or muscle atrophy.

3. The method according to claim 1 wherein the inhibitor of GLUD1 activates muscle satellite cells in the subject.

4. The method according to claim 1, wherein the muscle injury or muscle insult is caused by an ischemic or traumatic injury or insult.

5. The method according to claim 1, wherein the muscle wasting, muscle wasting disease, or muscle atrophy is sarcopenia.

6. The method according to claim 1, wherein the muscle wasting, muscle wasting disease, or muscle atrophy is associated with cachexia, cancer, AIDS, coeliac disease, chronic obstructive pulmonary disease (COPD), multiple sclerosis (MS), arthritis, rheumatoid arthritis (RA), congestive heart failure, tuberculosis (TBC), familial amyloid polyneuropathy, mercury poisoning (acrodynia), Crohn's disease, untreated/severe type 1 diabetes mellitus, anorexia nervosa, hormonal deficiency, frailty syndrome, spinal muscle atrophy, stroke, steroid therapy, poliomyelitis, spinal cord injury, hypercatabolic disease, or myotonia congenital.

7. The method according to claim 1, wherein the inhibitor of GLUD1 is a small compound inhibiting GLUD1, a nucleic acid based inhibitor of GLUD1, a biopharmaceutical compound inhibiting GLUD1, a GLUD1 knock-out macrophage, or a macrophage conditionally expressing a GLUD1 inhibitor.

8. The method according to claim 7, wherein the nucleic acid based inhibitor is a GLUD1-selective nucleic acid based inhibitor selected from a gapmer, a shRNA, a siRNA, an artificial microRNA, a dsRNA, an anti-sense oligomer, a ribozyme, a morpholino, a locked nucleic acid, a peptide nucleic acid, a Zinc-finger nuclease, a TALEN, a CRISPR-Cas, a CRISPR-C2c2, and a meganuclease.

9. The method according to claim 7, wherein the nucleic acid based inhibitor of GLUD 1 is an RNA based inhibitor of GLUD1.

10. The method according to claim 7, wherein the small compound inhibiting GLUD1 is selected from the group consisting of R162, purpurin, aurintricarboxylic acid, hexachlorophene, GW5074, bithionol, CK2 inhibitor, BSB, leoidin, erythrosin B, metergoline, diethylstilbestrol, calmidazolium, BH3I-2, suloctidil, ethaverine hydrochloride, epigallocatechin 3,5,-digallate, epigallocatechin 3-monogallate, epicatechin 3-monogallate, epigallocatechin, epicatechin, gallic acid, dimethyl-α-ketoglutarate, hymecromone methyl ether, strophanthidin, allopurinol, 5,7-dihydroxy-methylcoumarin, a compound according to any of Formulas I-VI, and GLUD1-inhibiting analogues of any of the foregoing compounds; or selected from the group consisting of a prodrug, co-crystal, polymorph and salt of any of the foregoing compounds or analogues.

11. (canceled)

12. An isolated GLUD1 knock-out macrophage, or an isolated macrophage conditionally expressing a GLUD1 inhibitor.

13. A pharmaceutical composition comprising an isolated GLUD1 knock-out macrophage or an isolated macrophage conditionally expressing a GLUD1 inhibitor, and an excipient.


SEQUENCE LISTING (6 sequences)

SEQ ID NO: 1 - DNA, 3051 bp, Homo sapiens
gggcaacccgcgcgggacccttcctccctagtcgcggggagtctgagaaagcgcgcctgtttcgcgaccatcacgcacctcccctccgcttgtggccatgtaccgctacctgggcgaagcgctgttgctgtcccgggccgggcccgctgccctgggctcggcgtccgccgactcggccgcgttgctgggctgggcccggggacagcccgccgccgccccgcagccggggctggcattggccgcccggcgccactacagcgaggcggtggccgaccgcgaggacgaccccaacttcttcaagatggtggagggcttcttcgatcgcggcgccagcatcgtggaggacaagctggtggaggacctgaggacccgggagagcgaggagcagaagcggaaccgggtgcgcggcatcctgcggatcatcaagccctgcaaccatgtgctgagtctctccttccccatccggcgcgacgacggctcctgggaggtcatcgaaggctaccgggcccagcacagccagcaccgcacgccctgcaagggaggtatccgttacagcactgatgtgagtgtagatgaagtaaaagctttggcttctctgatgacatacaagtgtgcagtggttgatgtgccgtttgggggtgctaaagctggtgttaagatcaatcccaagaactatactgataatgaattggaaaagatcacaaggaggttcaccatggagctagcaaaaaagggctttattggtcctggcattgatgtgcctgctccagacatgagcacaggtgagcgggagatgtcctggatcgctgatacctatgccagcaccatagggcactatgatattaatgcacacgcctgtgttactggtaaacccatcagccaagggggaatccatggacgcatctctgctactggccgtggtgtcttccatgggattgaaaatttcatcaatgaagcttcttacatgagcattttaggaatgacaccagggtttggagataaaacatttgttgttcagggatttggtaatgtgggcctacactctatgagatatttacatcgttttggtgctaaatgtattgctgttggtgagtctgatgggagtatatggaatccagatggtattgacccaaaggaactggaagacttcaaattgcaacatgggtccattctgggcttccccaaggcaaagccctatgaaggaagcatcttggaggccgactgtgacatactgatcccagctgccagtgagaagcagttgaccaaatccaacgcacccagagtcaaagccaagatcattgctgaaggtgccaatgggccaacaactccagaagctgacaagatcttcctggagagaaacattatggttattccagatctctacttgaatgctggaggagtgacagtatcttactttgagtggctgaagaatctaaatcatgtcagctatggccgtttgaccttcaaatatgaaagggattctaactaccacttgctcatgtctgttcaagagagtttagaaagaaaatttggaaagcatggtggaactattcccattgtacccacggcagagttccaagacaggatatcgggtgcatctgagaaagacatcgtgcactctggcttggcatacacaatggagcgttctgccaggcaaattatgcgcacagccatgaagtataacctgggattggacctgagaacagctgcctatgttaatgccattgagaaagtcttcaaagtgtacaatgaagctggtgtgaccttcacatagatggatcatggctgacttcctcactatcctcttcacatgtaacttctgcagacctatcacaagtttacatgtaaccacagaaatccctttctctcctgactcattaataatggataccattctcaacaagtcaatccaagtcagcccgttaaggagaaagaaattaaggttagcggatcatgtacaagctgagtgtgaaagtagaaatcacctacaccagagagccattttggtattttgcctttaaataaaaagcctcctttatctggctgtgcagccttgctctgtggcttttcccaacacaatcagtgctagtgctggggaggaacagtcaagagcagtcagttgcttgcttattttttctggatgagtctgggacacactgtaactttaacacatttaagaagtaggtgtgtggccttttcagaaggtggcatggtcctcaagtgagttcttagtattttatatcagcaaaataattcaattttgcaggttgcaaacaaatataaaacctgtttctgtttatgaatattattcttttagaatagaataagtacatgctgctgtaataaaattgcctttaatcacttaacaagcctaaccttgactcaaacagtgaatgcctatagaaataataaatgaaaaaaactagtatttttatatcataaaacaatgtcatttatagcttatcattcatgtattgtccagcagacattaaaagccctgtggataattaagttatcttcatacctgcaaaatggtggaggctattttcattaaaactgtcagaatttgcttaccataattatgatacagtccaaagaatgcagtcactttttatcatgttaactaattgttctcttttgaagatctatggttgactaattaaacaataattcaagtagagtgtcccagaaaaaaaccacttgggctccctgtttggagtctggctggctctgagcattgccaatggcccctactcacctgactttgtatcctctccttttagaggctttgcattctgcacccagcttcactaacagtgggctgaaaacatccttgggttgagtgtttcatttgggagttatttggccagggccttttgaacagtagtgtccccatgaagtgctagataatatatgtgtaagagtcagcttttttttttttttttaactctaacacccttcagaaatttctaactactttgtaactgcatggcttaacctggtgataaaagcagttattaaaagtctacgttttccaaaaaaaaaaaaaaa

SEQ ID NO: 2 - protein, 558 aa, Homo sapiens
MYRYLGEALLLSRAGPAALGSASADSAALLGWARGQPAAAPQPGLALAARRHYSEAVADREDDPNFFKMVEGFFDRGASIVEDKLVEDLRTRESEEQKRNRVRGILRIIKPCNHVLSLSFPIRRDDGSWEVIEGYRAQHSQHRTPCKGGIRYSTDVSVDEVKALASLMTYKCAVVDVPFGGAKAGVKINPKNYTDNELEKITRRFTMELAKKGFIGPGIDVPAPDMSTGEREMSWIADTYASTIGHYDINAHACVTGKPISQGGIHGRISATGRGVFHGIENFINEASYMSILGMTPGFGDKTFVVQGFGNVGLHSMRYLHRFGAKCIAVGESDGSIWNPDGIDPKELEDFKLQHGSILGFPKAKPYEGSILEADCDILIPAASEKQLTKSNAPRVKAKIIAEGANGPTTPEADKIFLERNIMVIPDLYLNAGGVTVSYFEWLKNLNHVSYGRLTFKYERDSNYHLLMSVQESLERKFGKHGGTIPIVPTAEFQDRISGASEKDIVHSGLAYTMERSARQIMRTAMKYNLGLDLRTAAYVNAIEKVFKVYNEAGVTFT

SEQ ID NO: 3 - DNA, 20 bp, Artificial Sequence
  gRNA
aatccctatcgattcctgtg

SEQ ID NO: 4 - DNA, 20 bp, Artificial Sequence
  gRNA
gaacagtcgcgtttgcgact

SEQ ID NO: 5 - DNA, 21 bp, Artificial Sequence
  Primer
aatccctatcgattcctgtgg

SEQ ID NO: 6 - DNA, 20 bp, Artificial Sequence
  Primer
gaaccggctgatgtgtttgg